(19) United States
(12) Patent Application Publication
(10) Pub. No.: US 2025/0331921 A1
(43) Pub. Date: Oct. 30, 2025

(54) SYSTEMS AND METHODS FOR LYMPH NODE ASSESSMENT

(71) Applicant: INTUITIVE SURGICAL OPERATIONS, INC., Sunnyvale, CA (US)

(72) Inventors: Hui Zhang, San Jose, CA (US); Ruchi C. Bhatt, San Jose, CA (US); Edward Boeschenstein, Pleasanton, CA (US); Shiyang Chen, Sunnyvale, CA (US); Energy Cruse, II, Foster City, CA (US); Akshaya Shanmugam, Santa Clara, CA (US); Jing Yu, San Francisco, CA (US); Reza Navid, Los Gatos, CA (US)

(21) Appl. No.: 18/865,176

(22) PCT Filed: May 12, 2023
(86) PCT No.: PCT/US2023/022078

§ 371 Date: Nov. 12, 2024

Related U.S. Application Data
(60) Provisional application No. 63/341,958, filed on May 13, 2022.

(51) Int. Cl. A61B 34/10; A61B 34/00; A61B 34/20; A61B 90/00

(52) U.S. Cl. CPC A61B 34/10; A61B 34/20; A61B 34/25; A61B 90/36; A61B 2034/105; A61B 2034/107; A61B 2034/2061; A61B 2034/254; A61B 2090/376

(57) ABSTRACT

A system for providing navigational guidance for a medical procedure may comprise a processor and a memory operably coupled to the processor for storing instructions that, when executed by the processor, cause the system to perform operations. The operations may comprise receiving an anatomic model of an anatomic region, performing an analysis of the anatomic model to facilitate selection a plurality of nodal sites for analysis, generating a procedure sequence for the plurality of nodal sites, providing navigational guidance to direct a medical instrument to a nodal site of the plurality of nodal sites in the procedure sequence, and gathering local image data from the medical instrument at the nodal site.

### CROSS-REFERENCED APPLICATIONS

This application claims priority to and benefit of U.S. Provisional Application No. 63/341,958, filed May 13, 2022 and entitled “Systems and Methods for Lymph Node Assessment,” which is incorporated by reference herein in its entirety.

### TECHNICAL FIELD

The present disclosure is directed to systems, methods, and computer program products for lymph node assessment and staging to determine nodal metastasis.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomic passageways and navigated toward a region of interest within a patient anatomy. Navigation may be assisted using images of the anatomic passageways. Improved systems and methods are needed to plan and safely perform lymph node assessment and staging to evaluate nodal metastasis.

### SUMMARY

The embodiments of the invention are best summarized by the claims that follow the description.

In one example, a system for providing navigational guidance for a medical procedure may comprise a processor and a memory operably coupled to the processor for storing instructions that, when executed by the processor, cause the system to perform operations. The operations may comprise receiving an anatomic model of an anatomic region, performing an analysis of the anatomic model to facilitate selection a plurality of nodal sites for analysis, generating a procedure sequence for the plurality of nodal sites, providing navigational guidance to direct a medical instrument to a nodal site of the plurality of nodal sites in the procedure sequence, and gathering local image data from the medical instrument at the nodal site.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure. The drawings should not be taken to limit the disclosure to the specific embodiments depicted but are for explanation and understanding only.

FIGS. 1A-1C illustrate lymph node stations of a thoracic region of a patient, according to some examples,

FIG. 2 is a flow diagram illustrating a method for planning and/or performing a lymph node clinical intervention.

FIGS. 3A-3B, 4A-4D, 5A-5B, 6A-6B, 7A-7B, 8 illustrate graphical user interfaces used to generate a procedural sequence for imaging and/or performing a clinical intervention at a plurality of lymph nodes.

FIGS. 9A-9B, 10A-10B, 11A-11B illustrate graphical user interfaces used to provide navigational guidance to navigate a medical instrument to the plurality of lymph nodes in the procedural sequence.

FIGS. 12A-12D, 13A-13B, 14A-14B, 15A-15B illustrate graphical user interfaces including intra-operative image data for assessment of the plurality of lymph nodes.

FIGS. 16A and 16B illustrate a graphical user interfaces displaying clinical intervention records stored for a plurality of the lymph nodes in the procedural sequence.

FIG. 17 is a schematic representation of a manipulator assembly, a medical instrument system, and an imaging system configured in accordance with various embodiments of the present technology.

FIG. 18 is a schematic representation of a portion of the medical instrument system of FIG. 17 extended within an anatomic region of a patient in accordance with various embodiments of the present technology.

### DETAILED DESCRIPTION

The present disclosure is directed to devices, systems, methods, and computer program products for lymph node assessment and staging to, for example, determine nodal metastasis. In some embodiments, for example, the assessment is performed on a cancer patient to determine the anatomic extent of cancer (“cancer staging”). The cancer staging process can include determining how much cancer is in the patient's body, where the cancer is located, and/or whether the cancer has spread from its original site (e.g., to the lymphatic drainage system, vasculature, other organ systems, etc.). For example, in the context of lung cancer (e.g., non-small cell lung cancer), a staging process can include imaging and biopsying multiple lymph nodes and/or lymph node stations (i.e., clinically-defined groupings of lymph nodes) near the airways of the lungs. Accurate staging is important for assessing patient prognosis and selecting the appropriate course of treatment. In some instances, for example, surgery may be recommended for early stage lung cancers, but may be contraindicated for later stage lung cancers. However, an operator performing a biopsy procedure may not be aware of clinically-recommended staging guidelines (e.g., which lymph nodes and/or lymph node stations should be biopsied), may not know how to apply the guidelines to the particular patient's pathology, and/or may not know how to perform the biopsy procedure efficiently while complying with the guidelines.

Accordingly, the present technology can aid an operator in planning and/or performing lymph node assessment by (i) identifying which lymph nodes and/or lymph node stations (collectively, “lymph node sites”) should be biopsied, and (ii) determining a sequence for biopsying the selected lymph node sites. In some embodiments, for example, a three-dimensional (3D) model of an anatomic region of a patient is generated (e.g., from preoperative image data) and segmented into components representing anatomic structures such as the airways, lungs, lymph nodes, vessels, and/or a target lesion. The segmented model can be used to select lymph node sites to be biopsied (e.g., based on the location of the target lesion, locations of the lymph node sites, lymphatic drainage pathways, clinical staging guidelines, etc.). The model can also be used to determine a sequence for biopsying the selected lymph node sites in an efficient manner while minimizing risk of cross-contamination. During the biopsy procedure, the selected lymph node sites and the biopsy sequence can be displayed to provide visual guidance to the operator and to facilitate navigation within the patient anatomy. The present technology is expected to increase operator compliance with clinical staging guidelines, as well as improve the efficiency and accuracy of the staging process, which may contribute to better patient outcomes.

The present technology is generally directed to planning and/or performing a medical procedure, such as a biopsy procedure for diagnosing a disease or condition of a patient. In some embodiments, for example, the systems described herein are configured to plan a biopsy procedure for staging a lung cancer (e.g., non-small cell lung cancer). The stages of lung cancer can be defined as follows:
Stage 0: The cancer has not spread from its original site (“in situ disease”).
Stage I: A small primary tumor located in only one lung that has not spread to any lymph nodes and has not metastasized.
Stage II: Either a larger primary tumor that has not spread to any lymph nodes or a smaller tumor in the lung that has spread to nearby lymph nodes.
Stage III: Cancer is found in the lung and in the lymph nodes in the middle of the chest (“locally advanced disease”). Stage III has two subtypes: IIIA (the cancer has spread only to lymph nodes on the same side of the chest where the cancer started) and IIIB (the cancer has spread to the lymph nodes on the opposite side of the chest and/or above the collar bone).
Stage IV: The cancer has spread to both lungs, to fluid in the area around the lungs, or to another part of the body, such as the liver or other organs (“advanced disease”).

As discussed above, accurate lung cancer staging may be important for assessing patient prognosis and/or determining the appropriate treatment options. For example, surgery may be recommended for patients with Stage 0 or Stage I cancer; treatment (e.g., chemotherapy, radiation therapy, radiochemotherapy, immunotherapy) followed by surgery may be recommended for patients with Stage II cancer; and treatment (e.g., chemotherapy, radiation therapy, radiochemotherapy, immunotherapy) without surgery may be recommended for patients with Stage III or Stage IV cancer.

In some embodiments, lung cancer staging involves obtaining tissue samples from one or more nodal sites within a thoracic region of the patient. As described above, the presence of cancer cells at certain nodal sites (e.g., lymph node stations in the middle of the chest, lymph node stations on the opposite side of the chest from the original cancer site, etc.) may correlate to more advanced stages of cancer. Accordingly, the extent and severity of the cancer can be assessed by systematically sampling lymph nodes from different lymph node stations in the thoracic region.

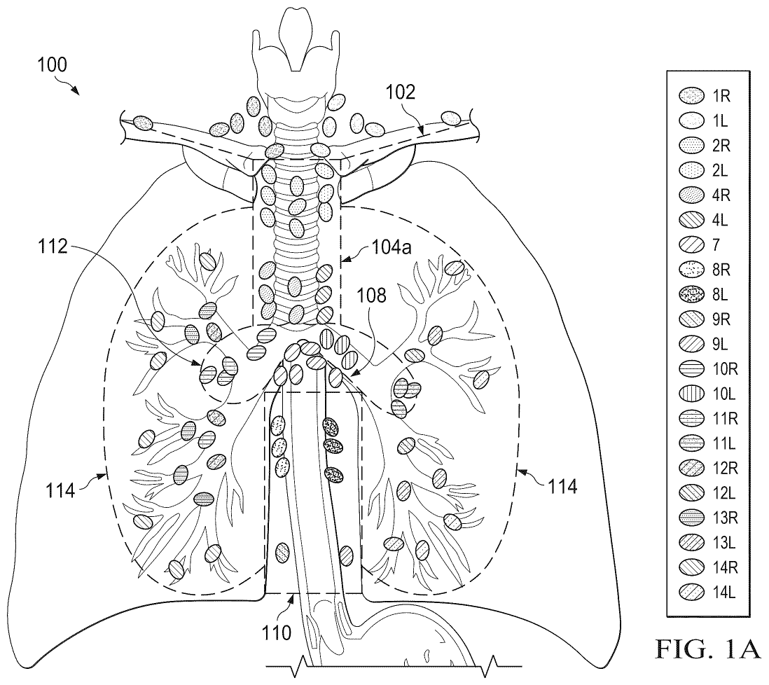

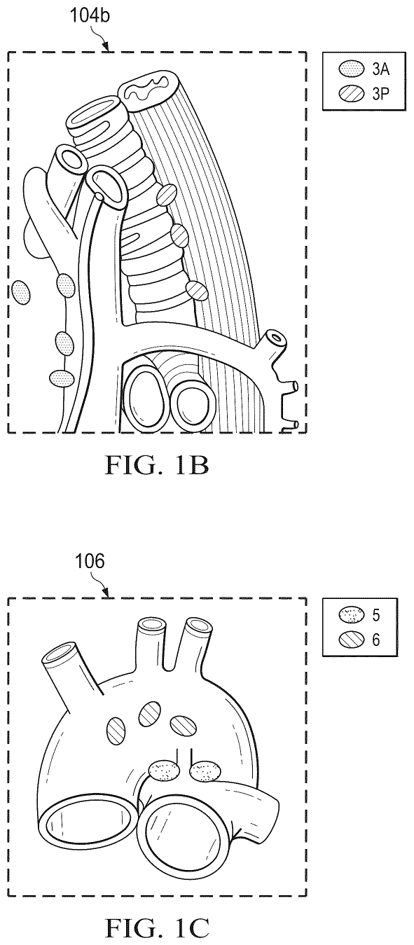

FIGS. 1A-1C illustrate lymph node stations of a thoracic region 100 of a patient. As can be seen in FIGS. 1A-1C and in Table 1 below, the lymph nodes of the thoracic region 100 can be grouped into 14 different lymph node stations (stations 1R-14). The lymph node stations can be grouped into 7 anatomic zones (102-114, indicated by broken lines in FIGS. 1A-1C).

TABLE 1
Thoracic Lymph Node Stations

Supraclavicular zone (102)
Station 1R: Right low cervical, supraclavicular,
and sternal notch lymph nodes
Station 1L: Left low cervical, supraclavicular,
and sternal notch lymph nodes
Upper zone (superior mediastinal lymph nodes) (104a, 104b)
Station 2R: Right upper paratracheal lymph nodes
Station 2L: Left upper paratracheal lymph nodes
Station 3A: Prevascular lymph nodes
Station 3P: Retrotracheal lymph nodes
Station 4R: Right lower paratracheal lymph nodes
Station 4L: Left lower paratracheal lymph nodes
Aortopulmonary zone (106)
Station 5: Subaortic lymph nodes
Station 6: Paraaortic lymph nodes
Subcarinal zone (108)
Station 7: Subcarinal lymph nodes
Lower zone (inferior mediastinal lymph nodes) (110)
Stations 8R, 8L: Paraesophageal lymph nodes
Stations 9R, 9L: Pulmonary ligament lymph nodes
Hilar and interlobar zone (pulmonary lymph nodes) (112)
Stations 10R, 10L: Hilar lymph nodes
Stations 11R, 11L: Interlobar lymph nodes
Peripheral zone (pulmonary lymph nodes) (114)
Stations 12R, 12L: Lobar lymph nodes
Stations 13R, 13L: Segmental lymph nodes
Stations 14R, 14L: Subsegmental lymph nodes

As described in greater detail below, the systems described herein can be configured to select one or more of the nodal sites shown in FIGS. 1A-1C and Table 1 to be biopsied during a medical procedure for staging lung cancer.

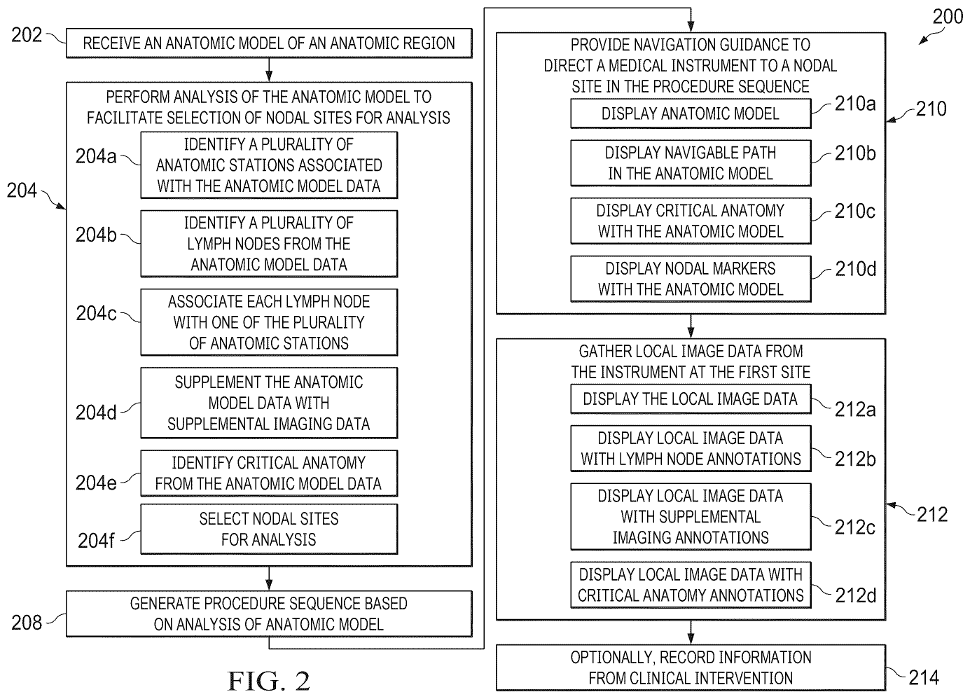

FIG. 2 is a flow diagram illustrating a method 200 for planning and/or performing a lymph node clinical intervention, such as a biopsy procedure, in accordance with various embodiments of the present technology. The method 200 is illustrated as a set of steps or processes 202-214. All or a subset of the steps of the method 200 can be implemented by a computing system or device, such as a workstation configured to perform preoperative planning for a medical procedure. Alternatively or in combination, all or a subset of the steps of the method 200 can be implemented by a control system of a medical instrument system or device, including various components or devices of a robotic or teleoperated system, as described in greater detail below. In some embodiments, one or more of the processes may be implemented, at least in part, in the form of executable code stored on non-transitory, tangible, machine-readable media that when run by one or more processors (e.g., the processing units of a control system) may cause the one or more processors to perform one or more of the processes. The methods described herein are illustrated as a set of operations or processes and are described with continuing reference to the additional figures. Not all of the illustrated processes may be performed in all embodiments of the methods. Additionally, one or more processes that are not expressly illustrated in may be included before, after, in between, or as part of the illustrated processes.

At a process 202, an anatomic model of an anatomic region may be received. Receiving the anatomic model may include generating the anatomic model, retrieving a stored anatomic model, obtaining an anatomic model from a networked source, or otherwise acquiring the anatomic model. The process 202 may include receiving image data of an anatomic region of a patient. The image data can include, for example, computed tomography (CT) data, magnetic resonance imaging (MRI) data, fluoroscopy data, thermography data, ultrasound data, optical coherence tomography (OCT) data, thermal image data, impedance data, laser image data, nanotube X-ray image data, and/or other suitable data representing the anatomic region where the biopsy procedure is to be performed. The image data can correspond to two-dimensional (2D), 3D, or four-dimensional (e.g., time-based or velocity-based information) images. In some embodiments, for example, the image data includes 2D images from multiple perspectives that can be combined into pseudo-3D images. The image data can be preoperative image data that is obtained before the biopsy procedure is performed on the patient. A 3D model of the anatomic region may be generated by segmenting the image data. The model can represent the anatomic region in which the biopsy procedure is to be performed (e.g., the airways of the patient's lungs), and can represent the locations, shapes, and connectivity of the passageways and other structures (e.g., lymph nodes, target lesion, vessels, etc.) within that region. In some embodiments, the model includes a plurality of segmented components corresponding to anatomic structures or features within the anatomic region. Examples of anatomic structures or features that may be included in the model include one or more of the following: airways (e.g., trachea, main carina, left main bronchus, right main bronchus, and/or sub-segmental bronchus), lymph nodes (e.g., any of the nodal sites described with respect to FIGS. 1A-1C and Table 1), vessels (e.g., aorta, superior vena cava, pulmonary trunk), lungs, and/or a target lesion (e.g., a tumor or other tissue site that is known or suspected to be cancerous).

The 3D model can be generated by segmenting graphical elements in the image data that represent or otherwise correspond to the anatomic structures or features. During the segmentation process, pixels or voxels generated from the image data may be partitioned into segments or elements and/or be tagged to indicate that they share certain characteristics or computed properties such as color, density, intensity, and texture. The segments or elements associated with anatomical features of the patient are then converted into a segmented anatomic model, which is generated in a model or image reference frame. To represent the model, the segmentation process may delineate sets of voxels representing the anatomic region and then apply a function, such as marching cube function, to generate a 3D surface that encloses the voxels. The model may be made by generating a mesh, volume, or voxel map. Additionally or alternatively, the model may include a centerline model that includes a set of interconnected line segments or points extending through the centers of the modeled passageways. Where the model includes a centerline model including a set of interconnected line segments, those line segments may be converted to a cloud or set of points. By converting the line segments, a desired quantity of points corresponding to the interconnected line segments can be selected manually or automatically. Various systems and methods for segmenting anatomic structures from image data are described in further detail in U.S. Patent Application Publication No. 2020/0030044 (filed Apr. 18, 2018) (disclosing a graphical user interface for planning a procedure); and U.S. Pat. No. 10,373,719 (filed Sep. 3, 2015) (disclosing systems and methods for pre-operative modeling); both of which are incorporated by reference herein in their entireties.

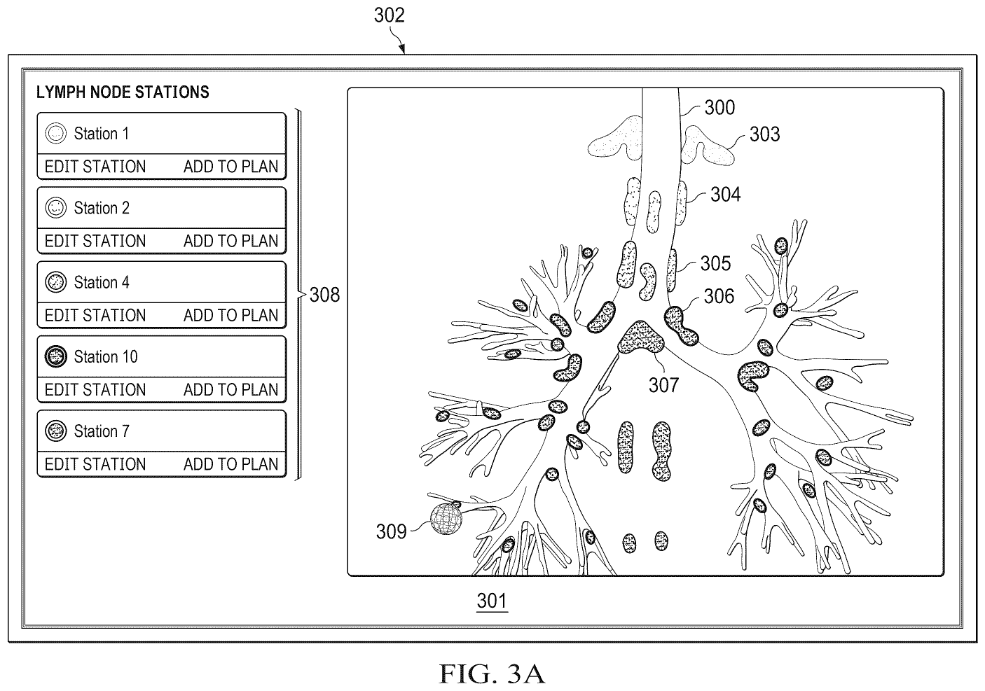

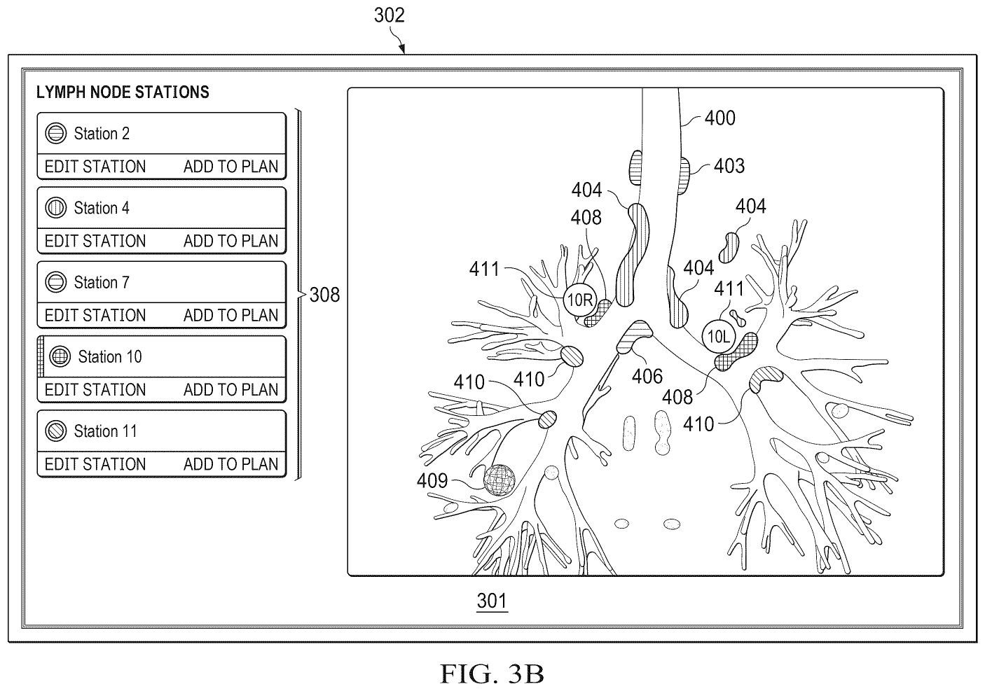

At a process 204, an analysis of the anatomic model may be performed. The analysis of the anatomic model may facilitate the selection of a plurality of nodal sites for analysis. The process 204 may, optionally, include one or more of the processes 204a-204f. At a process 204a, a plurality of node stations may be anatomically localized on the anatomic model. As described above, the lymph nodes of the thoracic region 100 can be grouped into 14 different lymph node stations. The anatomic model may be partitioned to correspond to the lymph node stations. With reference to FIG. 3A, in some examples, a branched anatomic model 300 including a target lesion 309 (e.g. a suspected lung tumor) may be displayed in a graphical user interface 301 on a display system 302. The lymph node stations may be indicated graphically on the user interface 301 such as by color, shading, borders, alphanumeric text or other graphical indicators of distinct node stations in the anatomic region. In the example of FIG. 3A, a Station 1 (303), a Station 2 (304), a Station 4 (305), a Station 10 (306), and Station 7 (307) may be illustrated as shaded regions in proximity to the anatomic model 300. The user interface 301 may allow a user to visualize the approximate location of the stations relative to the anatomic model. Interactive elements 308, such as menus, selectable elements, or information tabs associated with one or more of the displayed stations 303-307 may be displayed in the user interface 301.

FIG. 3B provides another example of a user interface 301 that may be used. In the example of FIG. 3B, a branched anatomic model 400 including a target lesion 409 (e.g. a suspected lung tumor) may be displayed in the graphical user interface 301 on the display system 302. The lymph node stations may be indicated graphically on the user interface 301 such as by color, texture, shading, borders, alphanumeric text or other graphical indicators of distinct node stations in the anatomic region. In the example of FIG. 3B, a Station 2 (403) may be indicated with a dark blue color region. A Station 4 (404) may be indicated with a red color region. A Station 7 (406) may be indicated with a light blue color region. A Station 10 (408) may be indicated with a yellow color region. Further differentiation of the Station 10 may be provided with textual markers (411) indicating the station number and a right or left side. A Station 11 (410) may be indicated with a green color region. The user interface 301 may allow a user to visualize the approximate location of the stations relative to the anatomic model. Interactive elements 308, such as menus, selectable elements, or information tabs associated with one or more of the displayed stations 403-410 may be displayed in the user interface 301 and may be color coded to match the colored regions of the stations 403-410.

At a process 204b, a plurality of lymph nodes may be identified in the anatomic model. The process 204b may include segmenting a plurality of lymph nodes in the image data. Segmenting of the lymph nodes can include, for example, analyzing the image data to identify graphical elements that correspond to lymph nodes, rather than other anatomic structures such as airways, vessels, etc. In some embodiments, lymph nodes are identified based on characteristics such as shape (e.g., oval or round, not tubular), size (e.g., approximately 1 cm in diameter), and/or location (e.g., near airways and/or within anatomic zones corresponding to lymph node stations). Once identified, the lymph nodes can be segmented into individual model components as discussed above.

The lymph node segmentation procedure can be performed in various ways, such as automatically (e.g., without requiring any operator input to identify the lymph nodes), semi-automatically (e.g., with some operator input), or manually by the operator. For example, automatic lymph node segmentation can be performed using a machine learning algorithm, such as a deep learning algorithm (e.g., a convolutional neural network or other type of neural network) that has been trained to identify and segment individual lymph nodes from CT scans or other image data of the patient anatomy. Training of the machine learning algorithm can be performed, for example, via supervised learning techniques using large sets of image data in which the lymph nodes have already been identified. Once trained, the machine learning algorithm can automatically recognize graphical elements in the image data that are likely to correspond to lymph nodes and can segment those graphical elements to create individual model components, as discussed above. Optionally, the machine learning algorithm can also be trained to automatically identify and segment other anatomic structures (e.g., airways, lesions, vessels, etc.).

A semi-automatic lymph node segmentation process can involve some steps that are performed automatically and some steps that are performed based on input from the operator. For example, the operator can select one or more locations in the image data that include lymph nodes and/or correspond to lymph node stations, and the computing system can analyze the selected locations to identify and segment the lymph nodes at those locations. In some embodiments, the operator provides input indicating the selected locations (e.g., via a suitable graphical user interface), such as by clicking or otherwise marking a point corresponding to a lymph node, drawing a boundary around edges or surfaces of a lymph node, selecting areas of the image data including lymph nodes and/or lymph node stations, or any other suitable process. The system can then use the input from the operator as a starting point for automatically detecting one or more lymph nodes in the image data. For example, the system may use edge detection algorithms, machine learning algorithms, etc. to search the image locations indicated by the operator for objects that are likely to correspond to lymph nodes. The results can be displayed to the operator for approval, rejection, modification, or other feedback. Alternatively or in combination, the system can automatically provide an initial selection of potential lymph nodes and the operator can accept, reject, or modify the selections. Optionally, if identification of individual lymph nodes is challenging (e.g., due to a low signal to noise ratio in the image data), the system and/or operator can instead identify and segment locations in the image data that correspond to lymph node stations (e.g., the lymph node stations and/or anatomic zones described above with respect to FIGS. 1A-1C), rather than identifying and segmenting individual lymph nodes.

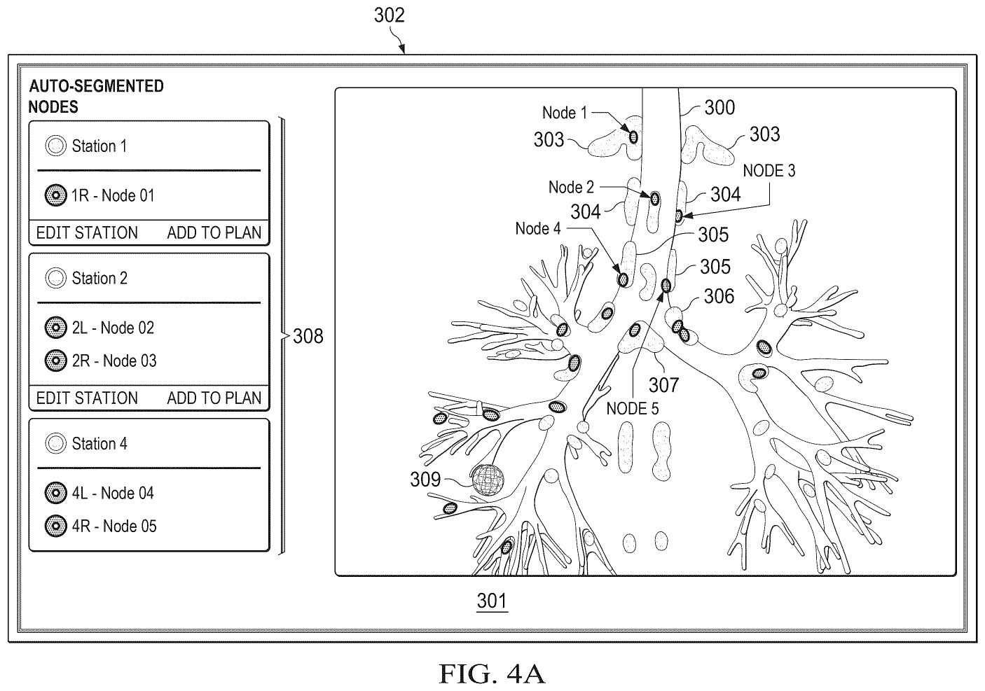

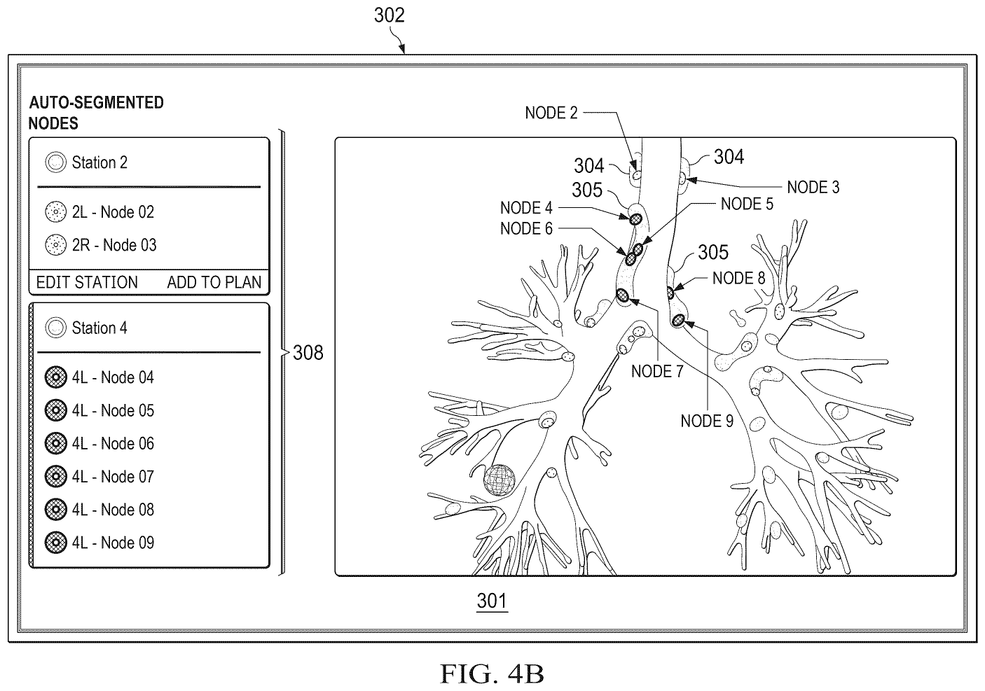

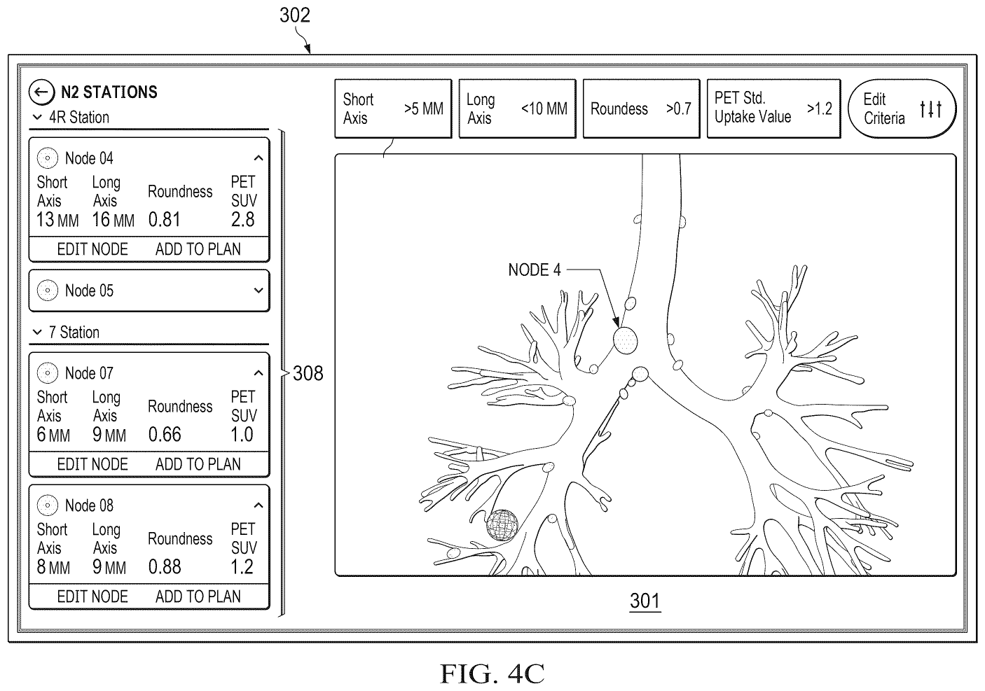

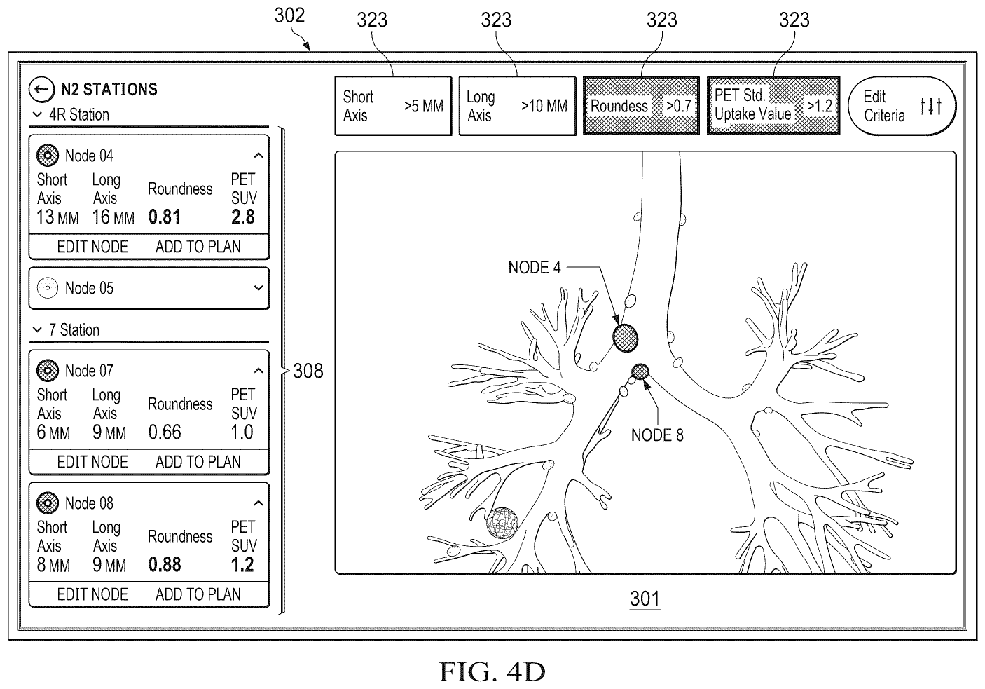

At a process 204c, one, a plurality of, or all of the identified lymph nodes may be associated with one of the plurality of anatomic stations. In some examples, the identified lymph nodes may be displayed as part of the anatomic model 300, providing a user with a visual indication of the nodes and the stations with which they are associated. For example, an image of the segmented lymph node may be overlayed on an image of an anatomic station of the anatomic model. With reference to FIG. 4A, each of the segmented lymph nodes may be assigned to and overlayed on a lymph node station (e.g., based on the location of the lymph node relative to other anatomic structures). For example, as shown in the user interface 301 of FIG. 4A, a Node 01 may be assigned to Station 1 (303). Nodes 02 and 03 may be assigned to Station 2 (304). Nodes 04 and 05 may be assigned to Station 4 (305). Other nodes may be assigned to stations in a similar manner. As shown in FIG. 4A, the interactive element 308 may include a displayed list view of the nodes grouped by assigned stations. The list view may include a size and/or a shape for the listed lymph nodes. The user interface 301 may include the segmented nodes (e.g. Nodes 01-05) overlayed on the respective stations (e.g. Stations 1, 2, and 4).

FIG. 4B provides another example of a user interface 301 that may be used. In the example of FIG. 4B, Nodes 02 and 03 may be assigned to Station 2 (304). Nodes 04-09 may be assigned to Station 4 (305). Interactive elements 308, such as menus, selectable elements, or information tabs associated with one or more of the displayed stations 304-305 may be displayed in the user interface 301, and nodes listed in the interactive element 308 may be color-coded or shaded to match the nodes displayed with the model.

FIG. 4C provides another example of a user interface 301 that may be used. In the example of FIG. 4C, segmented nodes may be displayed with characteristics derived from the image data (e.g. CT data). For example, a Node 04 may be displayed with the model with a size (e.g., short and/or long axis) and/or a shape (e.g. roundness) determined from CT image data. Further details of the derived characteristics may be displayed with the node information in the interactive element 308. For example, the dimensions of the long and short axis of the Node 04, a measure of the roundness of the Node 04, and a PET standardized uptake value (SUV) may be displayed. FIG. 4D provides the same user interface 301 as in FIG. 4C, but in this example, selected features of the nodes may be highlighted. The selected features may be selected by a clinician or may be selected by a control-system based on significance to the procedure or other criteria. In this example a Node 04 and a Node 08 displayed with the model may be highlighted with a color, brightness, or shading. The text describing the characteristics of interest in the interactive element 308 may also be highlighted with a color, brightness, or shading that may correspond to the displayed nodes. In this example, the roundness and PET SUV for Nodes 04 and 08 may be highlighted in the interactive element 308. In some examples, the nodes may be highlighted if a staging threshold is met. As shown in FIG. 4D, threshold elements 323 may be highlighted along with corresponding nodes if staging thresholds are met. For example, a staging threshold for the short axis may be a dimension greater than 5 mm. A staging threshold for the long axis may be a dimension less than 10 mm. A staging threshold for roundness may be greater than 0.7. A staging threshold for PET SUV may be greater than 1.2. In the example shown in FIG. 4D, Nodes 04 and 08 may be highlighted because their roundness exceed 0.7 and their PET SUV exceed 1.2. Node 07, on the other hand, might not be highlighted because its roundness and PET SUV do not exceed their respective thresholds. As another example, if only the short axis threshold is selected by a user or by a control system, Node 04, Node 07, and Node 08 may be highlighted in the GUI because their short axes exceed the short axis threshold.

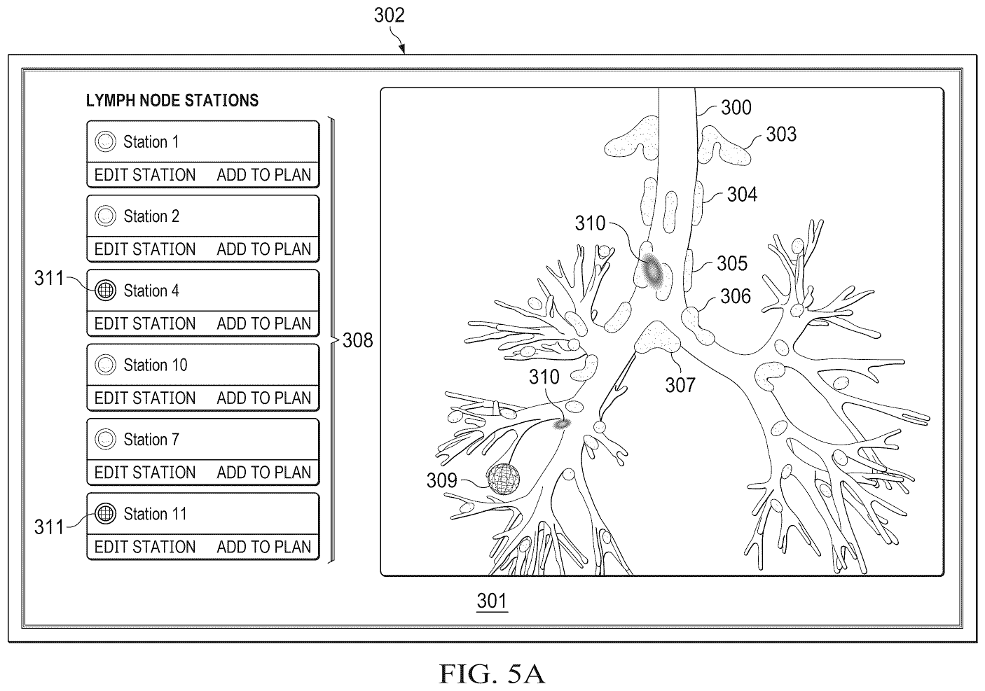

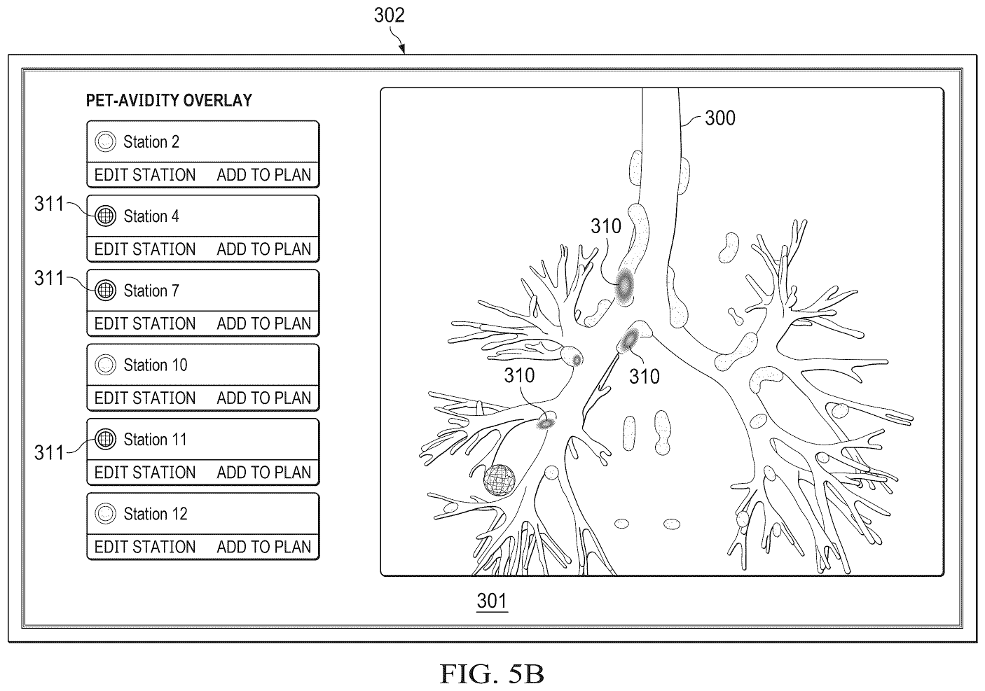

At a process 204d, supplemental imaging data may be used to augment the anatomic model data. In some examples, at process 204d, performing the analysis of the anatomic model may include overlaying imaging data such as positron emission tomography (PET) imaging data on the displayed anatomic model and lymph nodes. For example, supplemental imaging data in the form of flurodeoxyglucose (FDG)-PET imaging data may be generated for the relevant region of the patient anatomy. The FDG-PET imaging data may be used to augment the anatomic model data to provide additional information about metabolically active malignant areas in the imaged anatomic region. In some examples, the supplemental imaging data may be displayed as an overlay or integrated with the anatomic model. For example, with reference to FIG. 5A, areas 310 of high FDG avidity may be displayed as an overlay to the anatomic model 300 on the user interface 301. The areas of high FDG avidity may be associated with stations or specific nodes with the stations. For example the interactive elements 308 may provide information such as a highlighted clement 311 that indicate that Stations 4 and 11 are associated with activity in the supplemental imaging data that may indicate malignancy. FIG. 5B provides another example of a user interface 301 that may be used. In this example, three areas 310 of high FDG avidity may be displayed as an overlay to the anatomic model 300 on the user interface 301. The interactive elements 308 may provide information such as a highlighted elements 311 that indicate that Stations 4, 7, and 11 are associated with activity in the supplemental imaging data that may indicate malignancy.

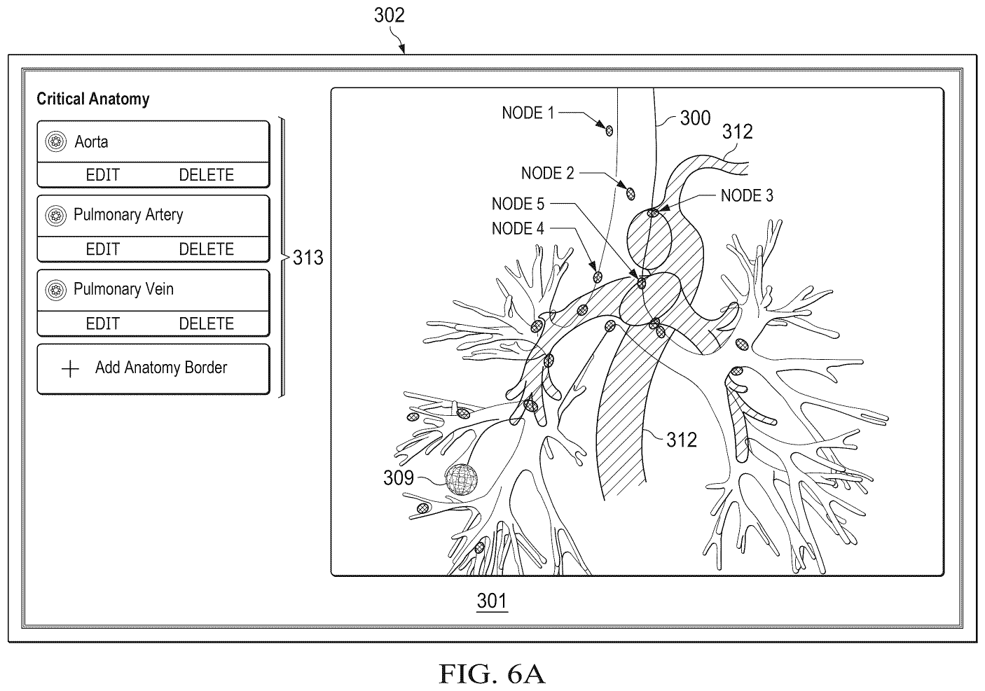

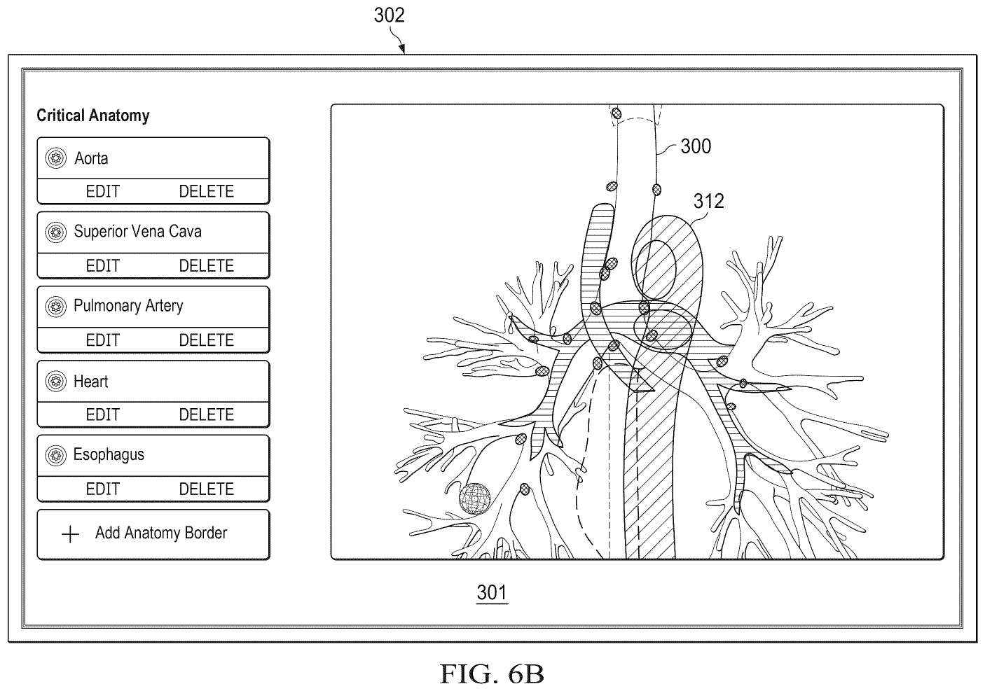

At a process 204c, critical or vulnerable anatomy may be identified from the anatomic image data. In some examples, at process 204c, performing the analysis of the anatomic model may further include displaying the critical anatomy that is located near at least one nodal site. Such critical or vulnerable tissue may include lung pleura, vasculature (e.g, aorta, pulmonary artery, pulmonary vein), the heart, and/or bullae in the region that should be avoided during an interventional procedure to prevent rupture or tearing. In some examples, the critical or vulnerable anatomy may be segmented from the anatomic image data and displayed as part of the anatomic model. For example, with reference to FIG. 6A, anatomy 312 may be displayed with the anatomic model 300 on the user interface 301. The displayed anatomy 312 may include graphical elements representing various anatomic structures such as vasculature (e.g., ascending aorta descending aorta, superior vena cava, pulmonary artery, pulmonary vein). The processes described above may be used to automatically, semi-automatically, or manually segment the image data to create a 3D model of the thoracic region. In some examples the display of the anatomy 312 may be toggled on or off depending on the stage of the assessment and the usefulness of the information to the viewer. Information 313 about the anatomy 312 may also be displayed on the user interface 301. FIG. 6B provides another example of a user interface 301 that may be used. In this example, the anatomy 312 may be color-coded. For example, arteries may be color-coded with red, and veins may be color-coded as blue.

At a process 204f, nodal sites may be selected for analysis. A nodal site may be a region that includes the location of a lymph node and may include areas proximate to the location of the lymph node, including a location in an anatomic passageway proximate to the location of a lymph node. As discussed above, selected nodal sites can be imaged, biopsied or otherwise analyzed to determine the stage of the patient's cancer, e.g., by assessing whether the cancer has spread from an initial site (e.g., a target lesion 309) to the nodal sites. For example, the cancer may be early stage cancer if nodal sites located near the target lesion test negative for malignant cells. Conversely, the cancer may be advanced cancer if nodal sites located away from the target test positive for malignant cells. The number of positive nodal sites may also correlate to the extent of the cancer, e.g., the cancer may be early stage if few or none of the biopsied nodal sites test positive and may be advanced stage if most or all of the biopsied nodal sites test positive. Accordingly, process 204f may involve selecting which nodal sites should be biopsied in order to accurately stage the patient's cancer. Process 204f may involve selecting one or more individual lymph nodes, one or more lymph node stations, or a combination thereof. In some embodiments, for example, one or more lymph node stations are selected without specifying any particular lymph nodes within that station to be biopsied. In other embodiments one or more specific lymph nodes can be selected, with or without specifying the corresponding lymph node stations.

The selection of the nodal sites can be based on the particular patient's pathology, such as the location of the target lesion in the patient. For example, process 204f may include selecting one or more nodal sites that are located downstream along the lymphatic drainage pathway of the target lesion (e.g., sentinel lymph nodes). The drainage pathways within the patient's anatomic region can be determined based on clinical guidelines or research, the patient's particular anatomy and physiology, and/or any other suitable considerations. For example, in the thoracic region, drainage pathways can generally proceed from lymph node stations at the peripheral portions of the lungs to the lymph node stations at the mediastinum between the lungs. Drainage pathways typically do not cross the lobe or to the other side of the peripheral lung. Accordingly, if the target lesion is located at the peripheral portion of the lung (e.g., near station 13R—FIG. 1A), process 204f may involve selecting some or all the nodal sites that are located between the target lesion and the mediastinum (e.g., stations 12R, 11R, and 10R—FIG. 1A), as well as mediastinal nodal sites (e.g., stations 4R and 7—FIG. 1A). Optionally, nodal sites that are located upstream from the target lesion (e.g., station 14R—FIG. 1A) and/or are not located along the drainage pathways from the target lesion (e.g., stations 14L, 13L, 12L—FIG. 1A) can be omitted. Drainage pathways may vary to some extent from patient to patient and may skip over lymph node stations and/or pass between lymph node stations. Accordingly, the selection process 204f may be customized to each patient's particular anatomy and/or other clinical considerations.

Alternatively or in combination, the nodal sites can be selected based on their location, such as their proximity to a target lesion (e.g. a lung tumor). Proximity may be assessed quantitively (e.g., based on the measured distance between the target lesion and the nodal site) and/or qualitatively (e.g., whether the nodal site is on the same side of the chest as the target lesion, in the middle of the chest, or on the opposite side of the chest as the target lesion). In some embodiments, process 204f may involve selecting nodal sites at different proximities to the target lesion. For example, the selection can include at least one nodal site on the same side of the chest as the target lesion (ipsilateral or “N1 nodes”), at least one nodal site in the middle of the chest (“N2 nodes”), and at least one nodal site on the opposite side of the chest as the target lesion (contralateral or “N3 nodes”). For example, if the target lesion is near station 13R (FIG. 1A), step 230 can involve selecting ipsilateral nodal sites (e.g., stations 12R, 11R, 10R, and 4R—FIG. 1A) and/or contralateral nodal sites (e.g., 12L, 11L, 10L, and 4L—FIG. 1A). Optionally, process 204f may involve selecting more nodal sites that are close to the target lesion (e.g., N1 nodes) and fewer nodal sites that are far from the target lesion (e.g., N2 nodes, N3 nodes). Nodal sites that are very far from the target lesion or otherwise unlikely to have metastasis can be excluded.

In some examples, the nodal sites may be selected based on predictive modeling. Predictive modeling, for example, can be used to predict which nodal sites are likely to have metastasis, based on the location of the target lesion, and can be performed using statistical models, machine learning models, or any other suitable technique. The predictive model can generate a risk score for each nodal site representing the probability that the cancer has metastasized to the particular site. Nodal sites associated with a higher risk score can be selected for biopsy, while nodal sites associated with a lower risk score can be excluded.

Alternatively or in combination, the nodal sites can be selected based on other parameters, such as one or more of the following: accessibility to the imaging or biopsy device (e.g., lymph nodes located near airways that are too narrow, too tortuous, or otherwise inaccessible to the biopsy device can be excluded), lymph node size (e.g., lymph nodes that are larger than 1 cm in diameter and/or larger than 5 mm in short-axis diameter can be selected), lymph node shape (e.g., lymph nodes that are abnormally-shaped can be selected), proximity to vulnerable anatomic structures (e.g., nodal sites that are too close to major blood vessels, lung pleura, large bullae, etc. can be excluded), spatial relationships between nodal sites and other anatomic structures (e.g., airways, lungs, lung nodules), patient-specific physiology, clinical guidelines or research (e.g., relating to cancer staging procedures), and the like.

Any suitable number and combination of nodal sites can be selected. In some embodiments, process 204f involves selecting at least one, two, three, four, five, or more different nodal sites to be biopsied (e.g., at least one, two, three, four, five, or more different lymph node stations). Optionally, process 204f may involve selecting a certain number of lymph nodes per station to be biopsied (e.g., at least one, two, three, or more lymph nodes). In some embodiments, certain nodal sites are always selected, such as the mediastinal lymph nodes (e.g., some or all of lymph node stations 2L, 2R, 3A, 3P, 4L, 4R, 8L, 8R, 9L, 9R—FIG. 1A).

The selection of the nodal sites can be performed automatically, semi-automatically, or manually. For example, the system can automatically analyze the locations of the target lesion, the lymph nodes, and/or other segmented anatomic structures in the 3D model and apply any of the selection parameters described above to select a subset of the lymph nodes for biopsy. The selection parameters can be determined by the system (e.g., encoded in the system software) or can be manually set by the operator (e.g., the operator can choose which selection parameters should be applied). Once the system has selected the nodal sites, the selection can be output to the operator for approval, rejection, or modification. Optionally, the operator can provide user input indicating which nodal sites should be biopsied (e.g., via a graphical user interface). For example, the operator can manually select certain anatomic zones or regions, and the system can subsequently identify and select nodal sites within those regions. The operator can also manually select specific nodal sites to be biopsied.

At a process 208, a procedure sequence for analyzing the selected nodal sites may be determined. The sequence can indicate the order in which the selected nodal sites should be imaged and/or biopsied during the procedure. The sequence of nodes and a path through the branched anatomic region may be determined based the locations of the lesion and/or lymph nodes, lymph flow direction, constraints on the navigating instrument (e.g., bend radius limitations) and/or travel efficiency (e.g., distance between successive sites and avoiding back-tracking with the instrument). In some embodiments, the sequence is configured to reduce the likelihood of cross-contamination between nodal sites (e.g., transferring malignant cells to a non-cancerous lymph node). Accordingly, the sequence can include biopsying nodal sites that are less likely to be positive for malignancy (e.g., sites that are located farther away and/or upstream from the target lesion) before nodal sites that are more likely to be positive (e.g., sites that are located closer to and/or downstream from the target lesion). The biopsy sequence may also include sampling peripheral nodal sites before central nodal sites.

In some embodiments, the sequence is based, at least partly, on a trajectory for navigating a medical instrument (e.g., an imaging device or a biopsy device) toward a selected plurality of nodes and/or target lesion. The trajectory can be a predetermined route that traverses anatomic passageways to reach the target lesion, and can be automatically, semi-automatically, or manually generated during preoperative planning for the procedure. In such embodiments, if the trajectory passes near one or more of the selected nodal sites, imaging may be performed or biopsy samples may be collected from those sites in the order they are encountered (e.g., as the biopsy device moves towards the target lesion). Alternatively or in combination, the sequence can also be determined based on any of the following considerations: reducing backtracking, reducing total distance traversed by the biopsy device, reducing total time for the biopsy procedure, avoiding passageways that are inaccessible to the biopsy device or otherwise difficult to navigate, and/or avoiding having the biopsy device pass through areas that are close to vulnerable anatomic structures (e.g., major blood vessels, lung pleura, large bullae).

In some examples, process 208 may also include generating at least one proposed path for navigating a biopsy device within the anatomic region to reach each of the sequenced nodal sites. The path can be configured to traverse the anatomic passageways between the sequenced nodal sites so that the medical instrument reaches the sites in the correct sequence simply by following the path. Optionally, the path can also include a route for navigating the medical instrument to the target lesion. The path can be generated in various ways, such as automatically, semi-automatically, or manually. For example, an operator can manually create some or all of the path by selecting passageways (e.g., airways) within the model via a suitable graphical user interface. Alternatively or in combination, some or all of the path can be generated automatically by the system. For instance, the system can use the model to identify and select passageways that are located close to the selected nodal sites and are accessible to the biopsy device (e.g., have a sufficiently large diameter). In some embodiments, the system automatically generates a proposed path, and the operator can either approve the path or manually revise the path (e.g., by adding, deleting, or otherwise modifying portions of the path). Conversely, the operator can manually create a path, and the system can automatically revise the path or propose revisions for approval by the operator.

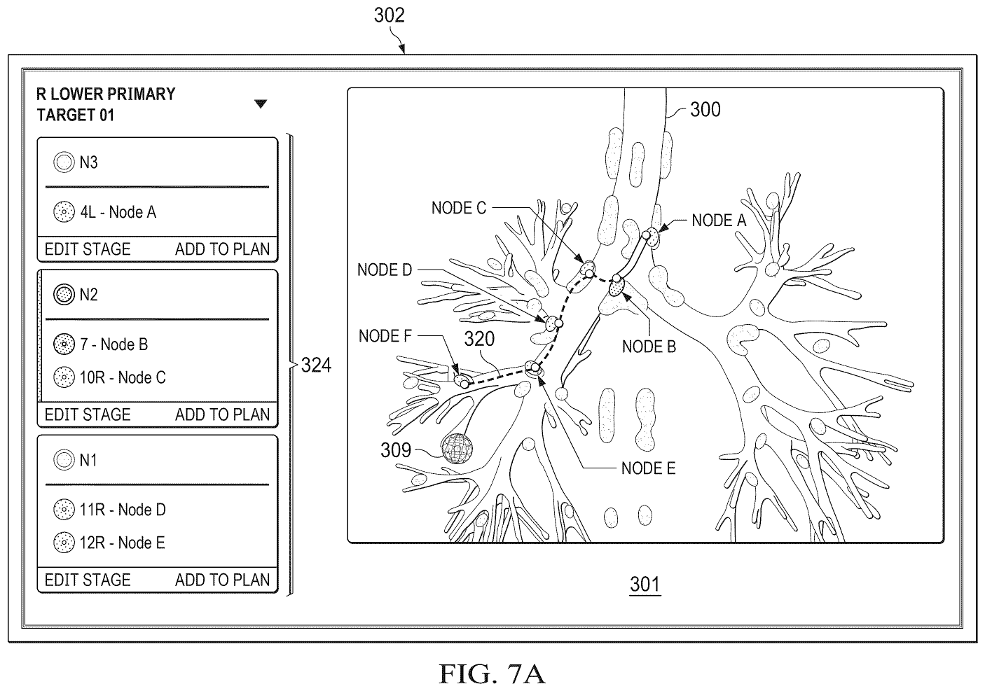

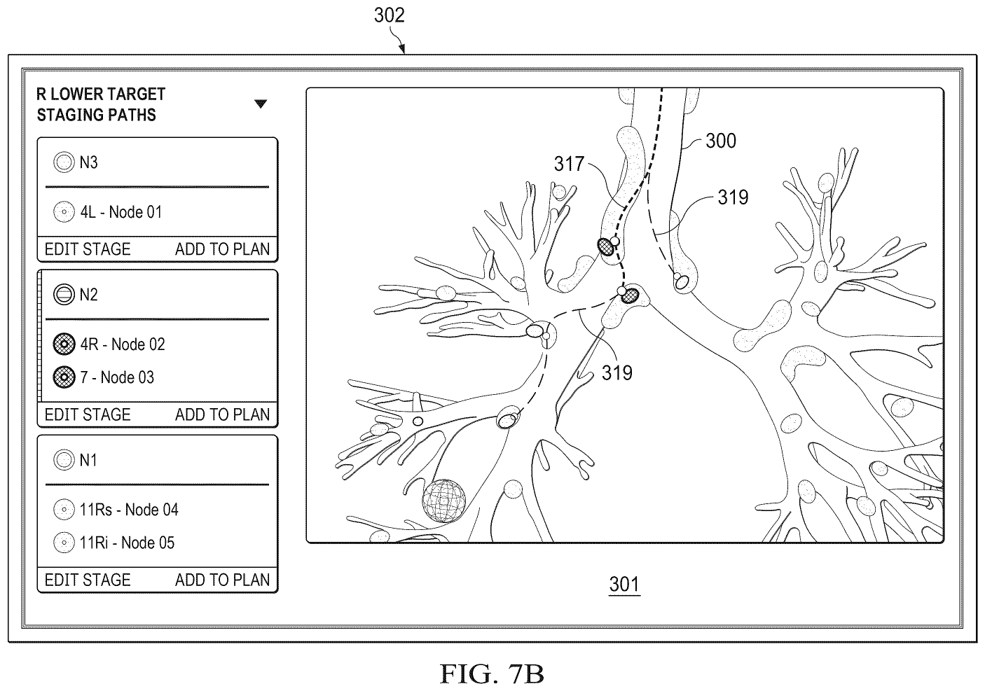

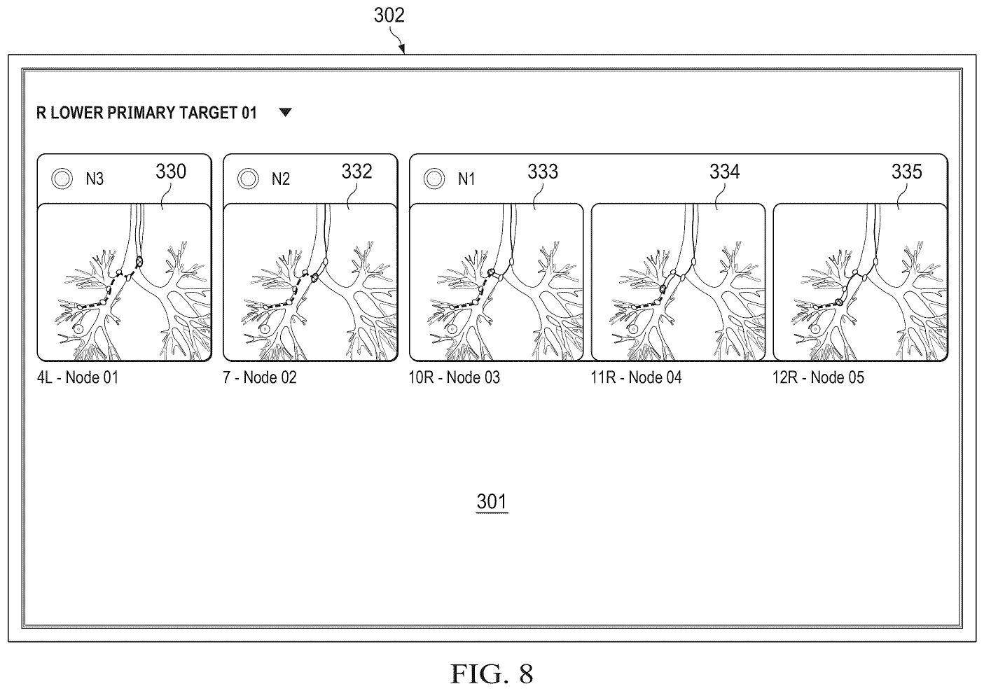

With reference to FIG. 7A, the user interface 301 may illustrate an ordered sequence of Nodes A-F and a navigation path 320 traversing anatomic passageways that extend near the sequenced nodes. The sequenced nodes A-F along the path 320 may be assigned to regions such as region N1, region N2, and/or region N3. The regions may be associated with a distance from the target lesion 309. For example, region N1 may be a region closest to the lesion 309 and region N3 may be farthest from the lesion 309. An information panel 324 may provide information about the sequenced nodes including the regions and stations to which the nodes are assigned. For example, Node A may be in Region N3, Nodes B and C may be in Region N2, and Nodes D and E may be in Region N1. In this example, paths 319 or path segments that have not yet been traversed may be indicated with a different color, dash pattern, or other distinguishing characteristic than paths 317 that have been traversed. For example, as shown in FIG. 7A, a first portion of the path between Nodes A and B may be a solid line indicating that portion of the path that has been reached by the medical instrument, and a second portion of the path between Nodes B and F may be a dashed line indicating that portion of the navigable path that has not been reached by the medical instrument. FIG. 7B provides another example of a user interface 301 that may be used. With reference to FIG. 8, in some examples, the user interface 301 may provide sequential images 330-334 that illustrate a step-by-step navigational path between the sequenced nodes.

Optionally, the navigation path may include an imaging location near each sequenced nodal site. The imaging location may provide a preferred vantage point from which to obtain intra-operative images (e.g., using ultrasound imaging technology) of the selected nodal site.

Optionally, the navigation path may include an exit location near each selected nodal site. The exit location can correspond to a point where a biopsy device exits the passageways to reach the nodal site (e.g., by puncturing through the lumen of the passageway at the exit location). For example, the exit location can be a point in the passageway that is closest to the site. The path can further include a path segment connecting the exit location to the nodal site (referred to herein as an “exit segment”). The length of the exit segment can be configured to be less than or equal to the maximum insertion depth of the biopsy device. For example, some biopsy needles may not be able to perform a biopsy of a target that is more than 3 cm from the exit location.

In some embodiments, the navigation path is configured to avoid one or more vulnerable anatomic structures, such as vessels, lung pleura, large bullae, etc. For example, puncturing the lung pleura during the biopsy procedure could cause pneumothorax and/or other conditions that are dangerous to the patient. Accordingly, the path, exit locations, and/or exit segments can be constrained to avoid vulnerable anatomic structures. This can be accomplished, for example, by defining one or more hazard fences surrounding the vulnerable anatomic structures that are used to denote locations that the path cannot contact and/or overlap. The hazard fences can be created automatically, semi-automatically, or manually by the operator. Additional techniques for creating paths within an anatomic region are described in further detail in U.S. Patent Application Publication No. 2020/0030044 (filed Apr. 18, 2018) (disclosing a graphical user interface for planning a procedure), which is incorporated by reference herein in its entirety. An image representing the location and shape of a traversing medical instrument 356 may also be displayed on the user interface 301.

The output of the process 208 (e.g., the 3D model, selected nodal sites, biopsy sequence, and/or path) can be saved (e.g., as one or more digital files) as part of a plan for the biopsy procedure. In embodiments where the plan is created on a preoperative planning workstation, the plan can be transferred to a medical instrument system that will be used to perform the intra-operative assessment of the selected nodal sites which may include local imaging and/or a biopsy procedure. Subsequent to the planning, during the intra-operative imaging and biopsy procedure, the 3D model, selected nodal sites, and/or biopsy sequence can be displayed to the operator (e.g., via a graphical user interface) to provide visual guidance and instructions for navigating to the selected biopsy sites, at described in greater detail below.

At a process 210, navigational guidance may be provided to direct a medical instrument to a nodal site in the procedure sequence. The nodal site may be a region that includes the location of a lymph node in the sequence of lymph nodes and may include areas proximate to the location of the lymph node, including a location in an anatomic passageway proximate to the location of a lymph node. In some examples, the instrument may be a medical instrument used to perform intra-operative imaging in the region of the first sequenced node, the last sequenced node, or any node in between the first and last sequenced nodes. In some examples, the instrument may be a medical instrument used to conduct a clinical intervention, such as a biopsy, with the first sequenced node. In some examples, the instrument may be configured to perform multiple functions including intra-operative imaging and biopsy. For example, the medical instrument may be an endobronchial ultrasound/transbronchial fine needle aspiration (EBUS-TBNA) instrument that may perform both ultrasound imaging at the first site and biopsying of the first lymph node in the sequence.

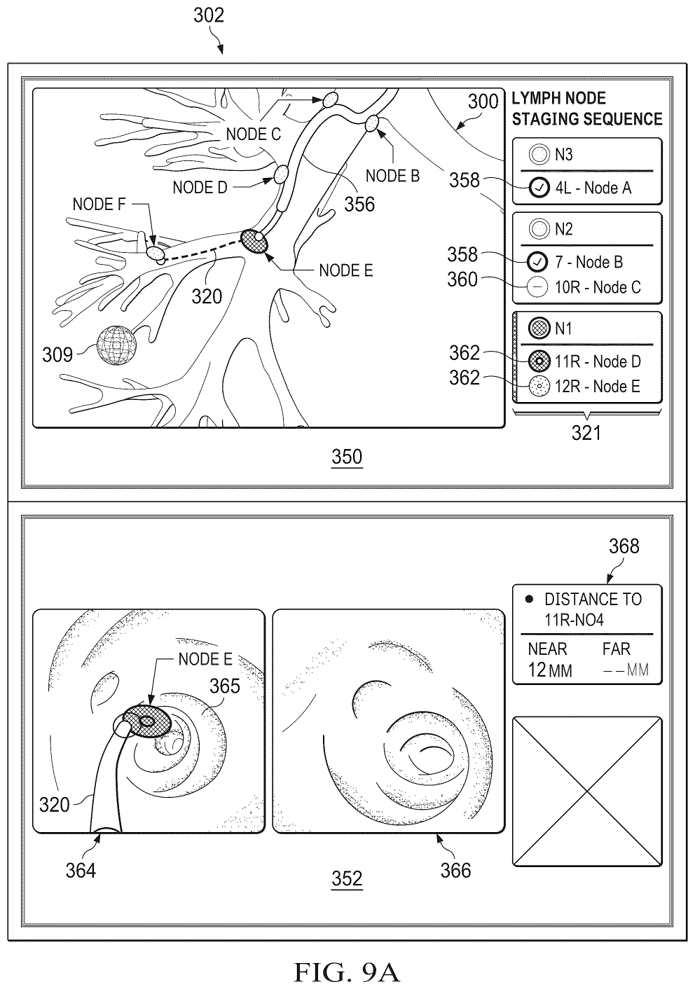

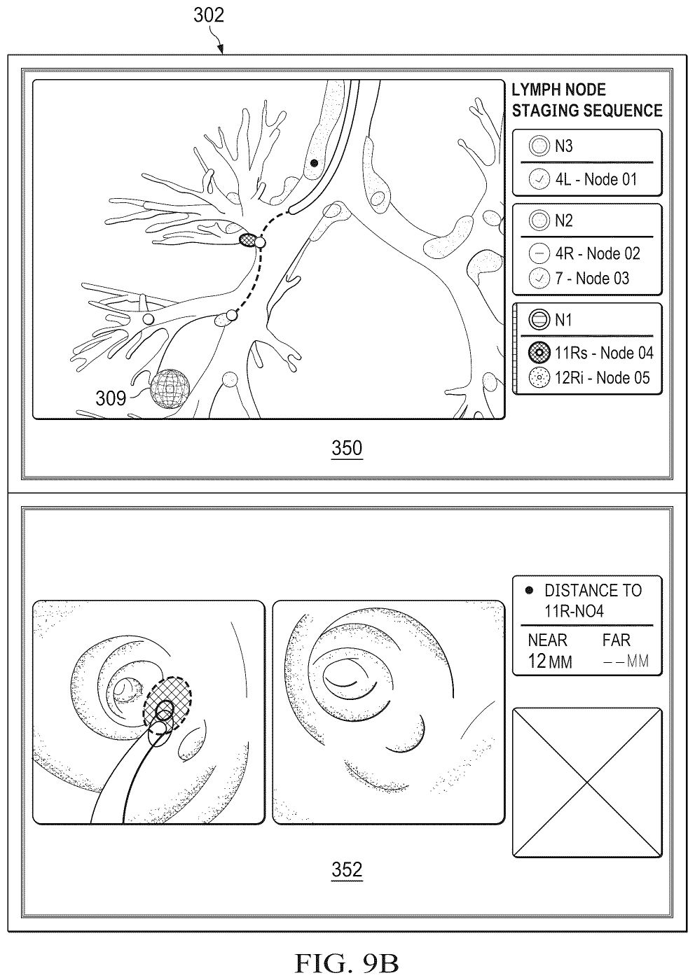

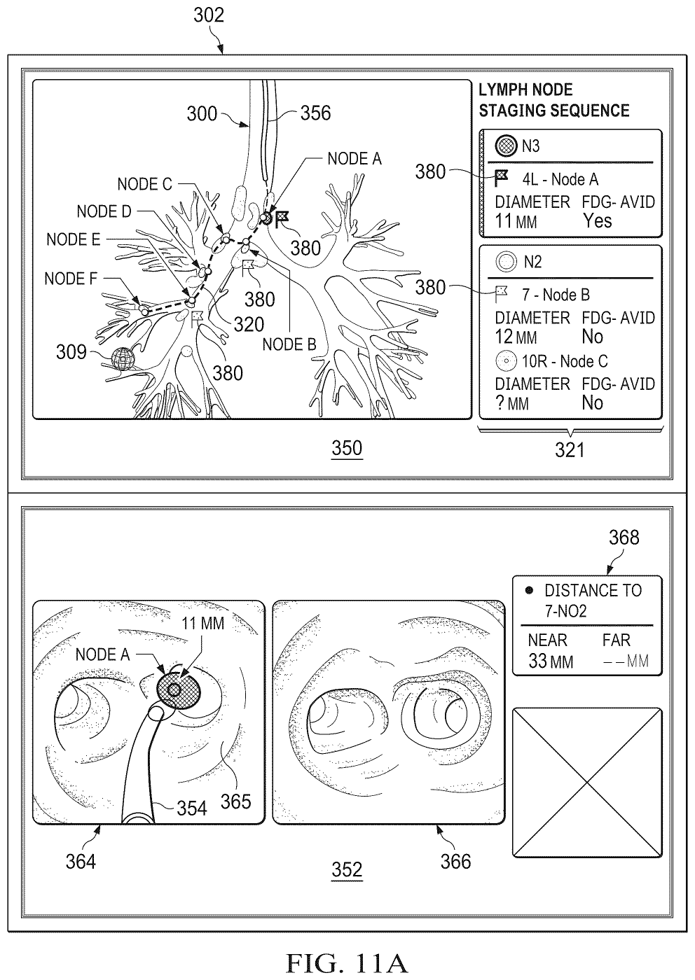

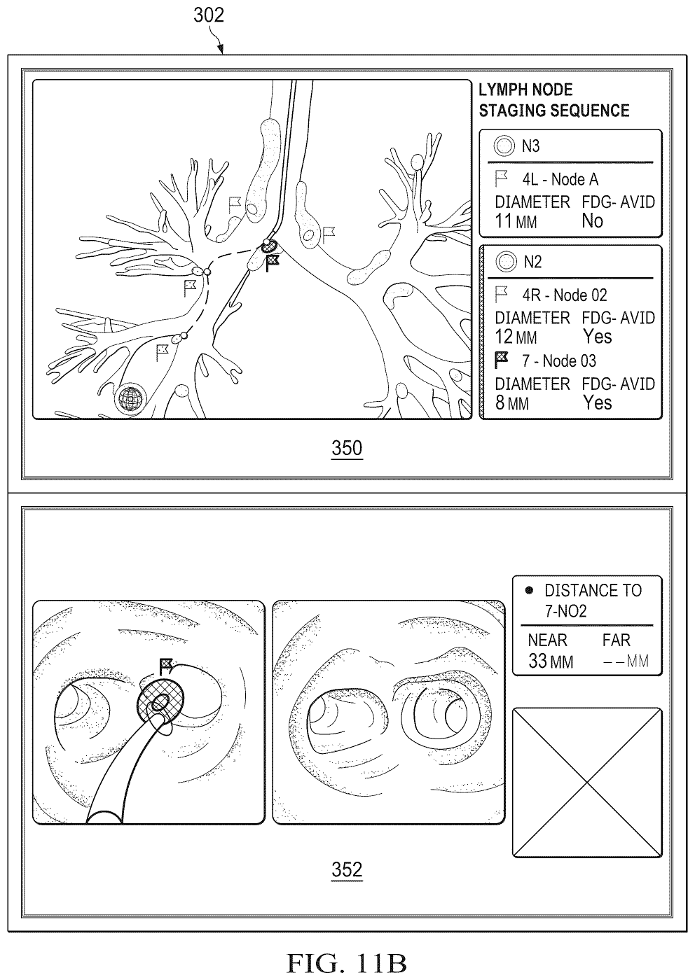

The process 210 may, optionally, include one or more of processes 210a-210d. At a process 210a, and with reference to FIG. 9A, providing navigational guidance may include displaying an image of the anatomical model. For example, the display system 302 may display at least a portion of the anatomic model 300. For example the anatomic model 300 may be displayed in a graphical user interface 350 in a first pane of the display system 302. As shown in FIG. 4A and 4B, providing navigational guidance may further include displaying images of segmented nodes over nodal stations and/or displaying interactive element 308, including the list view. With reference to FIG. 9A, the navigational guidance may also or alternatively include indicators that indicate if a nodal site is to be sampled by the medical instrument (e.g. node indicator 380 in FIG. 11A), if a nodal site has been sampled by the medical instrument (e.g. check mark indicator 358), and/or if a nodal site has not been sampled by the medical instrument (e.g. dash mark indicator 360). The navigational guidance may be registered and transformed to be displayed in any of various displayed views. For example, any of the disclosed navigational guidance may provided with a view of the three-dimensional image of the anatomic model (e.g. model 300), a virtual endoscopic image (e.g. virtual endoscopic image 364), and/or a real-time endoscopic image (e.g. real-time endoscopic image 366).

At a process 210b, and with further reference to FIG. 9A, providing navigational guidance may include displaying a representation of a navigable path through the procedure sequence for the plurality of nodal site. For example, the user interface 350 may display the navigable path 320 between the sequenced nodes. The user interface 350 may also include a representation of the medical instrument 356 along the path 320. The user interface 350 may also include the information panel 321 that provides information about the sequenced nodes including the regions and stations to which the nodes are assigned. During the navigation, the information panel 321 may also provide information about the procedure status. For example, a check mark 358 positioned next to a listed node may indicate that a procedure (e.g., an imaging procedure and/or a biopsy procedure) has been completed at the node. As another example, a dash mark 360 positioned next to a listed node may indicate that the node was omitted from the sequences and no procedure was performed. As another example, a dot mark 362 may indicate nodes in the sequence which have not yet been encountered by the instrument 356. In some examples, a graphical user interface 352 in a second pane of the display 302 may display a virtual endoscopic image 364 (e.g. a virtual fly-through image) of the model 300. The image 364 may display the anatomical wall 365, a location of the Node E toward which the instrument 356 is advancing, and the path 320. The display 302 may also display a real-time endoscopic image view 366 obtained by the medical instrument 356 within the anatomic passage. The display 302 may also display a navigation panel 368 that displays information about a distance from a distal end of the instrument 356 to the Node E. The display system 302 may be the same as the display system used for planning processes 202-208 or may be a different display system used by a clinician performing the clinical procedure. The graphical information displayed in the user interfaces 350, 352 may displayed in any configuration in one or more displayed panes on one or more display screens. Some of the graphical information may be selectively displayed based on user preference. FIG. 9B provides another example of a user interfaces 350, 352 that may be used.

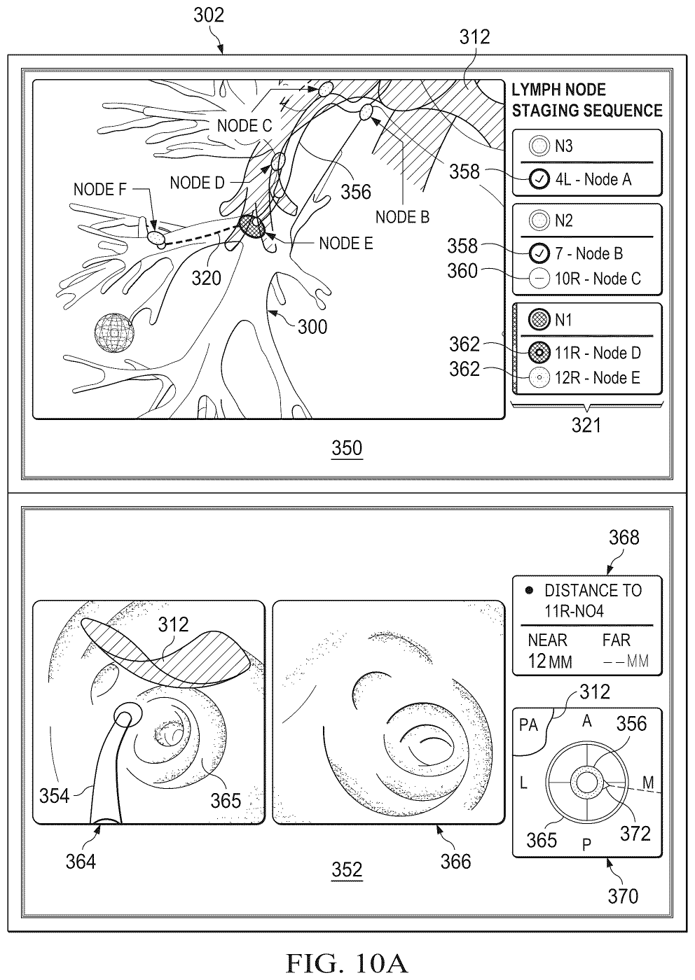

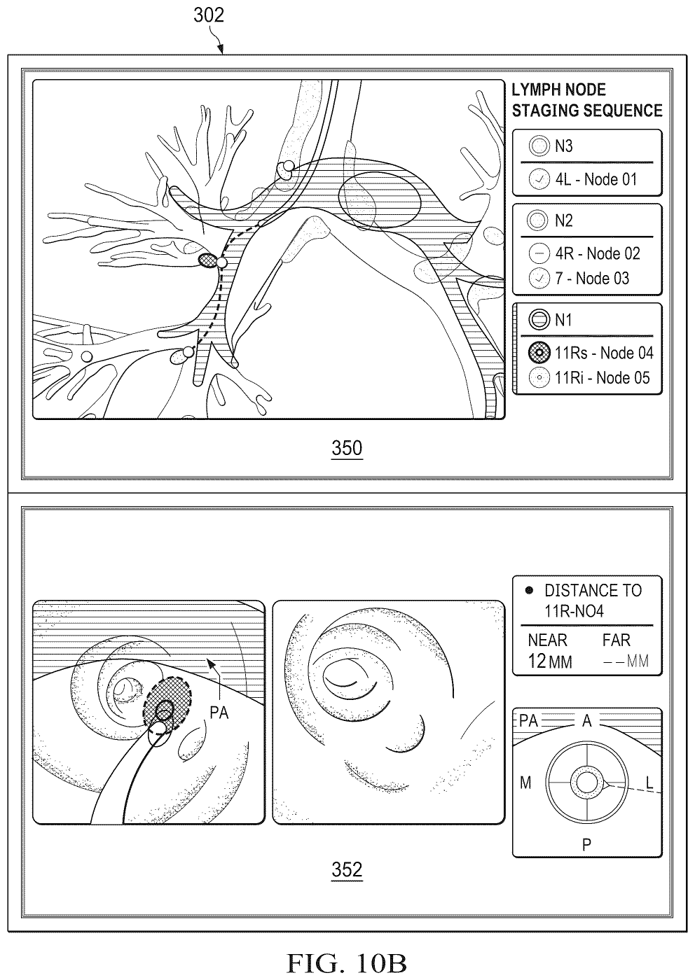

At a process 210c, and with reference to FIG. 10A, providing navigational guidance may include displaying a representation of critical anatomy along the navigable path. For example, an outline, semi-transparent illustration, or other graphical representation of critical or vulnerable anatomy 312 may be displayed with the model 300 and navigable path 320 in any of the user interface panels 350, 364, 366. The display of the representation of the anatomy 312 may toggled on or off to assist the navigating clinician with understanding the critical structures, such as vasculature, near to each node in the sequence. Navigational guidance may be provided by a user interface orientation clement 370 in the user interface 352 which may include a representation of the passageway wall 365, the instrument 356, the critical anatomy 312 and/or a direction indicator 372 of an imaging element (e.g. an ultrasound image transducer) carried by the instrument 356. FIG. 10B provides another example of a user interfaces 350, 352 that may be used.

At a process 210d, providing navigational guidance may include displaying a representation of nodal markers adjacent to one or more of the plurality of nodal sites. Nodal markers may be displayed with the anatomic model. Nodal markers may be used to designate identifying information about the associated node. For example, a nodal marker may indicate that a node has been imaged by the medical instrument. Additionally or alternatively, the nodal marker may indicate that a node should be biopsied based on an assessment of one or more sources of information (e.g. segmentation information, intra-operative ultrasound imaging, supplemental PET imaging). For example, with reference to FIG. 11A, the nodes (e.g. Nodes A, B, E) in the sequence may be marked to indicate they should be biopsied. The panel 321 may also include the nodal markers 380 associated with nodal information that has been generated, for example, from intra-operative imaging (e.g., size and composition information), clinical intervention with the node (e.g., biopsy information), graphical segmentation (e.g., size and distance information), and/or supplemental imaging (e.g. FDG avidity information). For example, the nodal information for marked Node A includes the diameter of the node (11 mm) and whether the node is FDG-Avid (Yes). FIG. 11B provides another example of a user interfaces 350, 352 that may be used. Characteristics of the nodal marker such as presence or absence, color, or direction may provide further indication that the node is intended to be sampled.

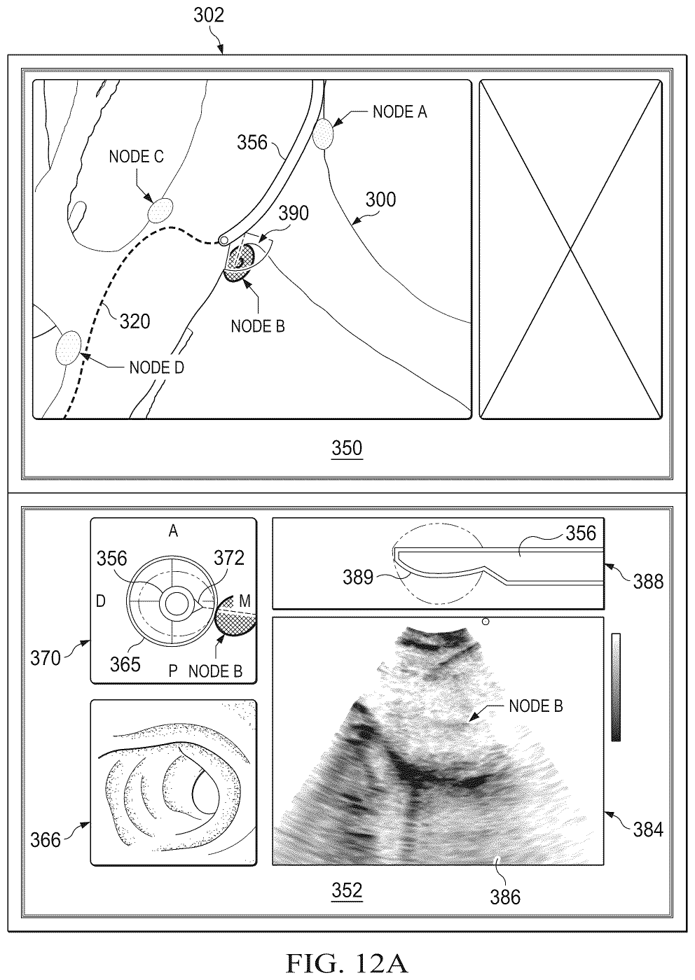

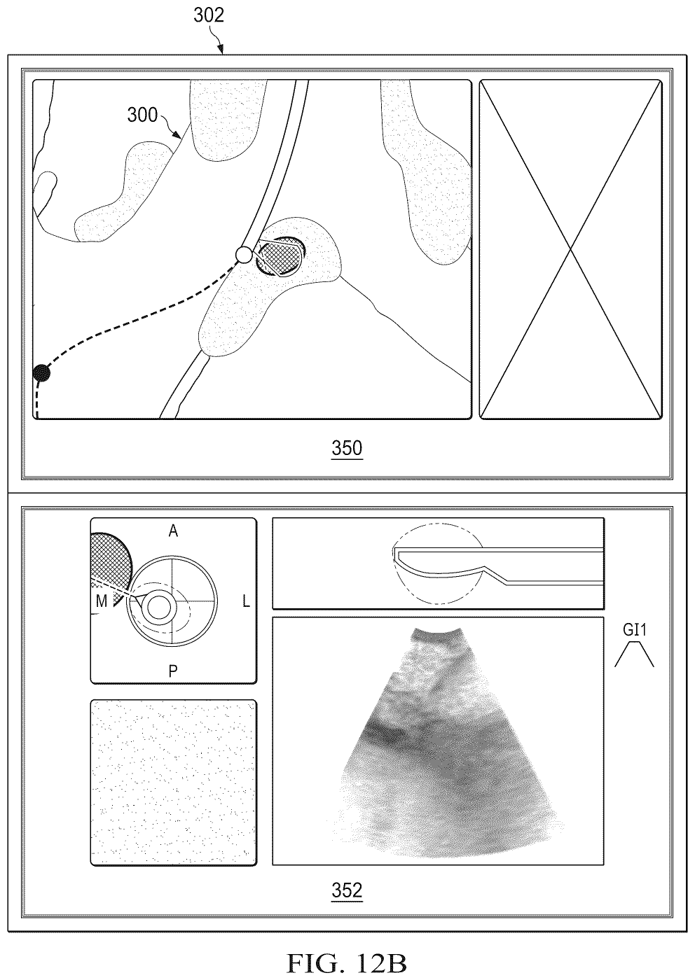

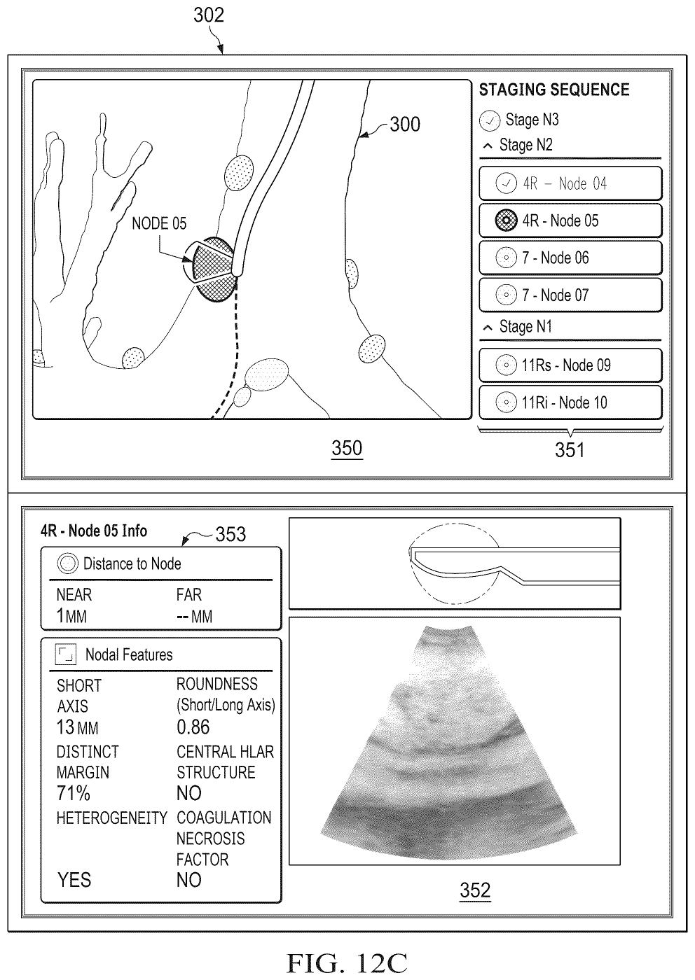

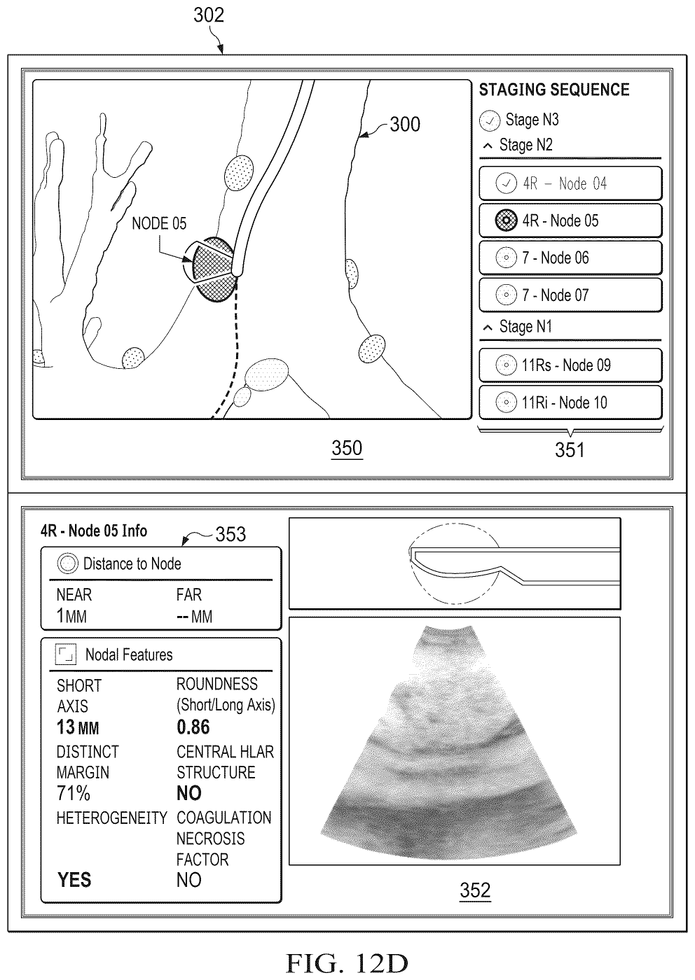

At a process 212, local image data may be gathered from the medical instrument positioned in the anatomic passageway near first nodal site. For example, the medical instrument may be an EBUS-TBNA instrument used to gather intra-operative ultrasound images of the lymph node at the first site. The process 212 may, optionally, include one or more of the processes 212a-212d. At a process 212a, the local image data may be displayed. For example, and with reference to FIG. 12A, the instrument 356 (e.g., an EBUS-TBNA instrument) may have a field of view area 390 that includes the Node B. The field of view area 390 may be a form of navigational guidance for the medical instrument. In the user interface 352, a panel 384 may display local image data as an ultrasound image 386 including Node B in of the field of view area 390. The image 386 may provide size, shape, and other information about the Node B that may be recorded and used for subsequent positioning/apposition and orientation of the same or a different biopsy instrument for biopsying the Node B. A panel 388 may provide a profile view of the instrument 356 indicating the direction of the transducer 389 with respect to the field of view area 390 displayed in ultrasound image 386. FIG. 12B provides another example of a user interfaces 350, 352 that may be used.

FIG. 12C provides another example of a user interfaces 350, 352 that may be used. In the example of FIG. 12C, a staging sequence window 351 may be provided in the user interface 350. The staging sequence window 351 may include a listing of the nodes in the staging sequence and may highlight (e.g. with a shading, brightness, or color) the current node (e.g. Node 05) in both the model 300 and the window 351. In this example, the user interface 352 may include a node information window 353 that may include imaging (e.g. CT and/or EBUS) derived characteristics of the nodes. For example, the node information window may include a distance to the node and nodal features such as a short axis dimension, a roundness, a measure of distinct margin, an indicator of central hilar structure, a heterogeneity indicator, and/or a coagulation necrosis factor. FIG. 12D may include the same user interfaces 350, 352 as in FIG. 12C but in this example, certain items of information (e.g. short axis, roundness, hilar indicator, heterogeneity indicator) may be highlighted based on user interest, significance to the procedure, or other criteria.

Various types of image annotations may be displayed in various combinations with the local image data (e.g. the ultrasound image 386). Additionally or alternatively, the image annotations may be registered and transformed to be displayed in any of various displayed views. For example, model annotations based on the transformed image annotations may be overlayed on an image of the anatomic model (e.g. three-dimensional model 300). For example, orientation annotations based on the transformed image annotations may be overlayed on a user interface orientation element (e.g. user interface orientation element 370). Similarly, annotations from other views may be transformed to be displayed with the local image data.

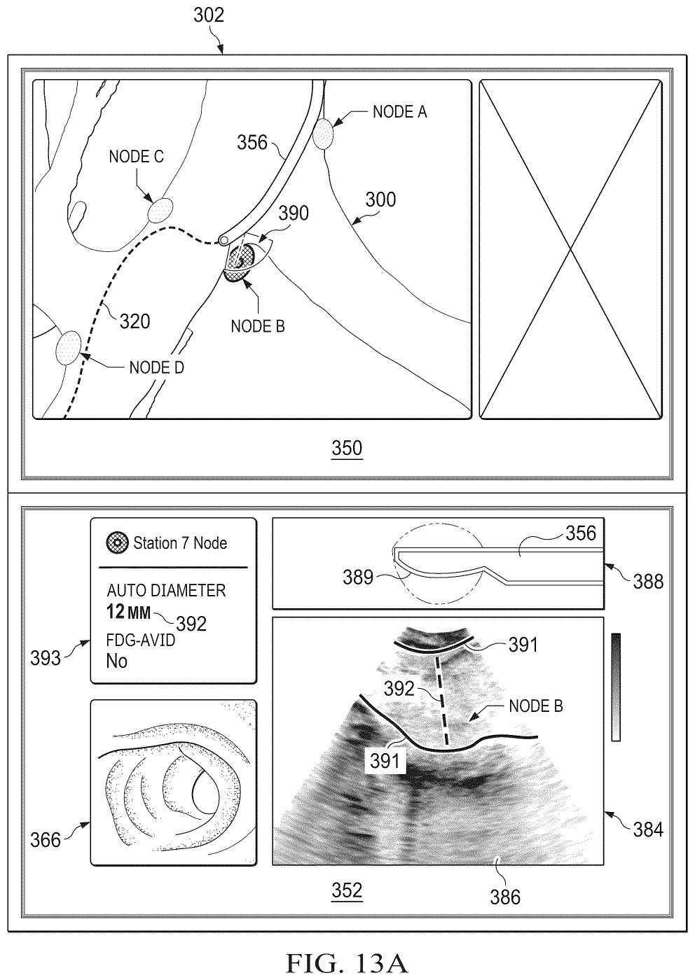

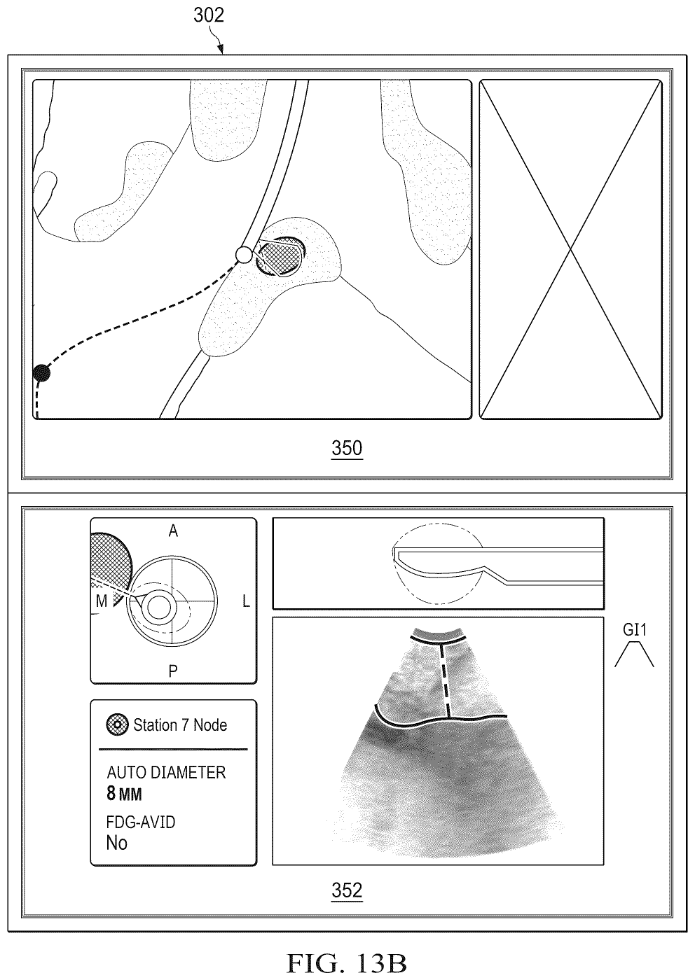

At a process 212b, the local image data may be displayed with the lymph node annotations. For example, and with reference to FIG. 13A, image annotations may include boundary annotations 391 that may be made by a clinician or by graphical analytical software to indicate the boundaries of the Node B. The boundary annotation may be overlayed on the ultrasound image 386. The image annotations may also or alternatively include a distance indicator 392 that may provide a size dimension for the Node B which may be displayed in a panel 393. The distance indicator 392 may provide a measured dimension between a near side and a far side of a lymph node visible in the ultrasound image 386. The distance indicator 392 may be overlayed on the ultrasound image 386. FIG. 13B provides another example of a user interfaces 350, 352 that may be used.

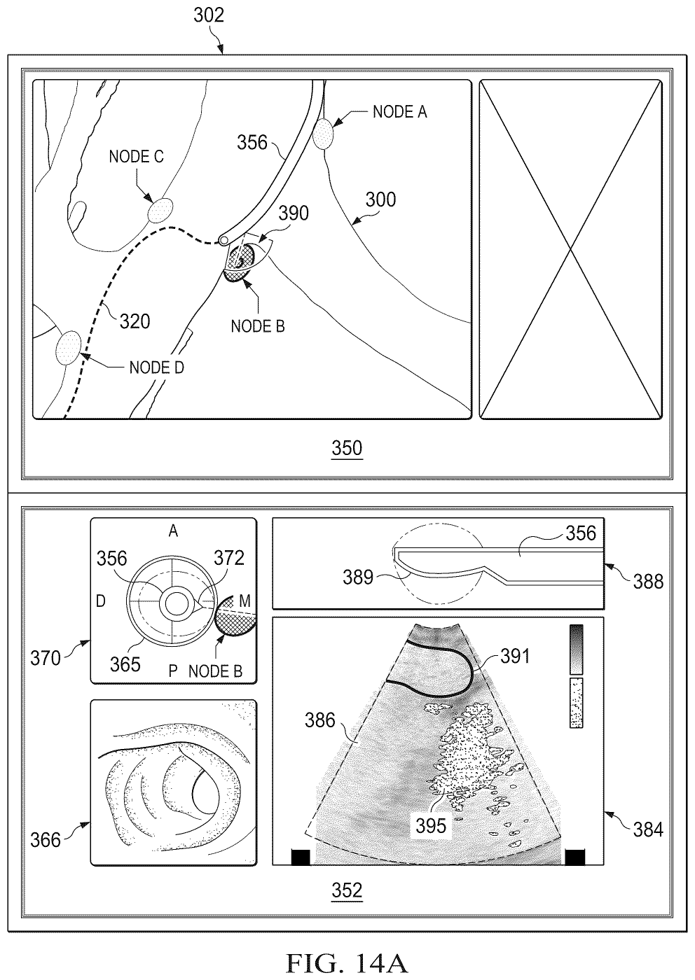

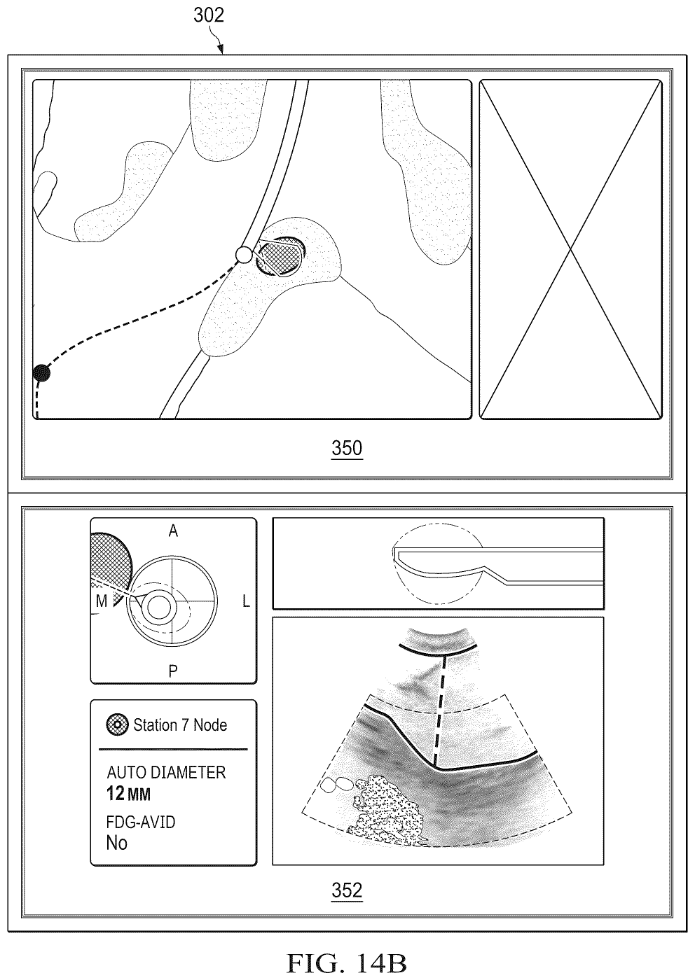

At a process 212c, the local image data may be displayed with supplemental imaging annotations. For example, and with reference to FIG. 14A, the image annotations may include doppler information 395 that may be overlayed on the ultrasound image 386 in the panel 384 to further annotate the image 386. FIG. 14B provides another example of a user interfaces 350, 352 that may be used.

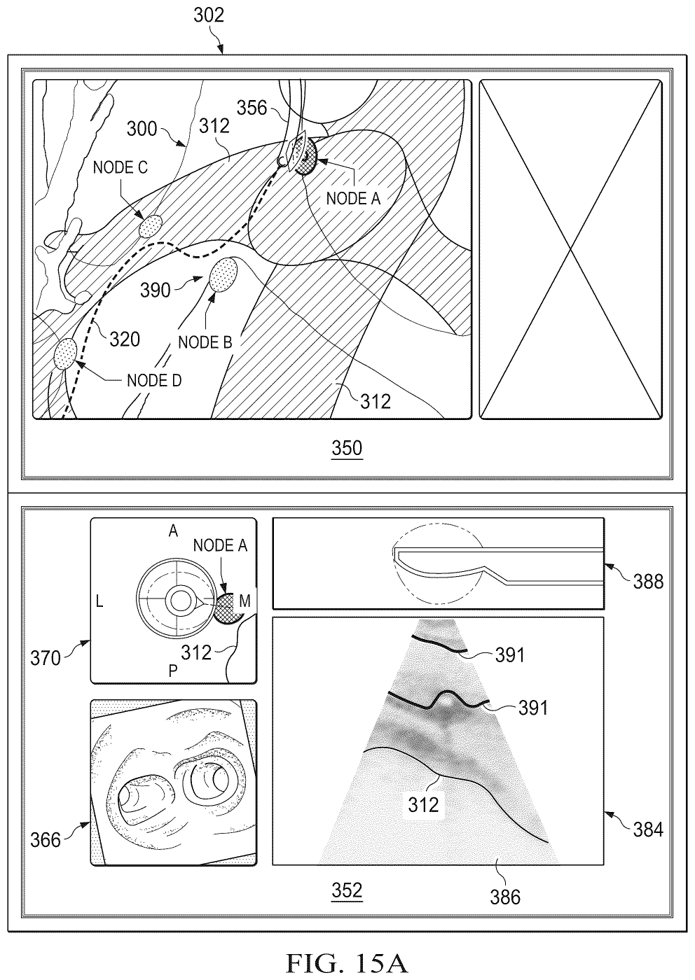

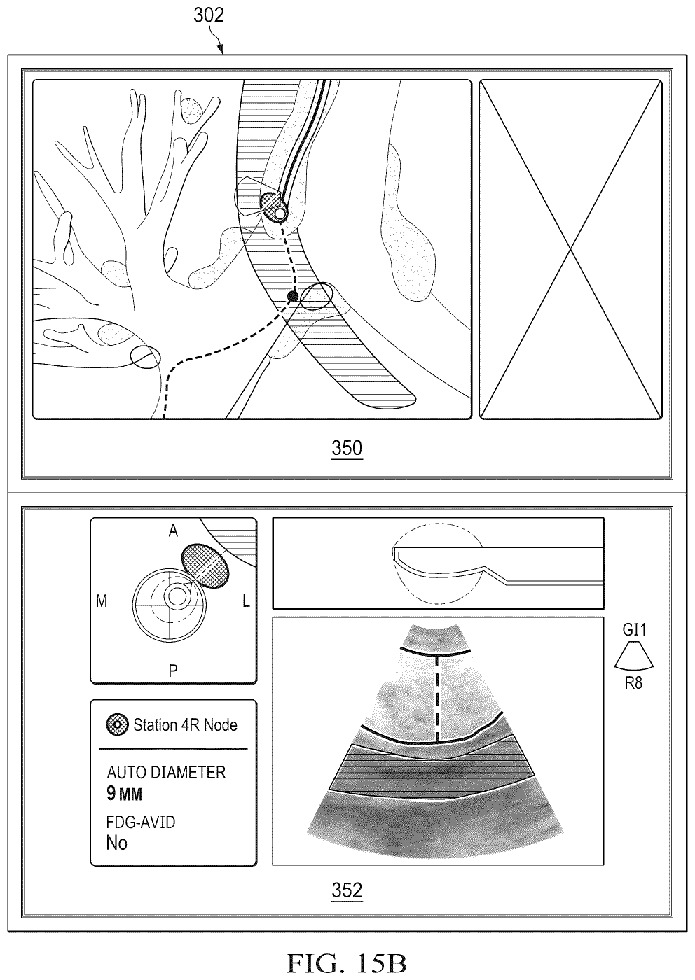

At a process 212d, the local image data may be displayed with the critical anatomy annotations. For example, and with reference to FIG. 15A, image annotations in the form of the critical anatomy indicator 312 and boundary annotations 391 for the Node A may be overlayed on the ultrasound image 386 in the panel 384. FIG. 15B provides another example of a user interfaces 350, 352 that may be used.

Any of the annotations, including the boundary annotations 391, the distance indicator 392, the doppler information 395 or the critical anatomy 312 may be interchangeably or selectively displayed or not displayed with ultrasound image 386, user interface element 370, and/or the anatomic model 300.

The processes 210, 212 may be repeated at each node in the sequence of lymph nodes. In some procedures, a clinical intervention such as a biopsy procedure may be performed immediately after the guidance and local imaging processes 210, 212 are performed at a nodal site, before advancing to the next node in the sequence. In some procedures, the guidance and local imaging processes 210, 212 may be performed for each node in the sequence before a clinical intervention is performed on any of the nodes in the sequence.

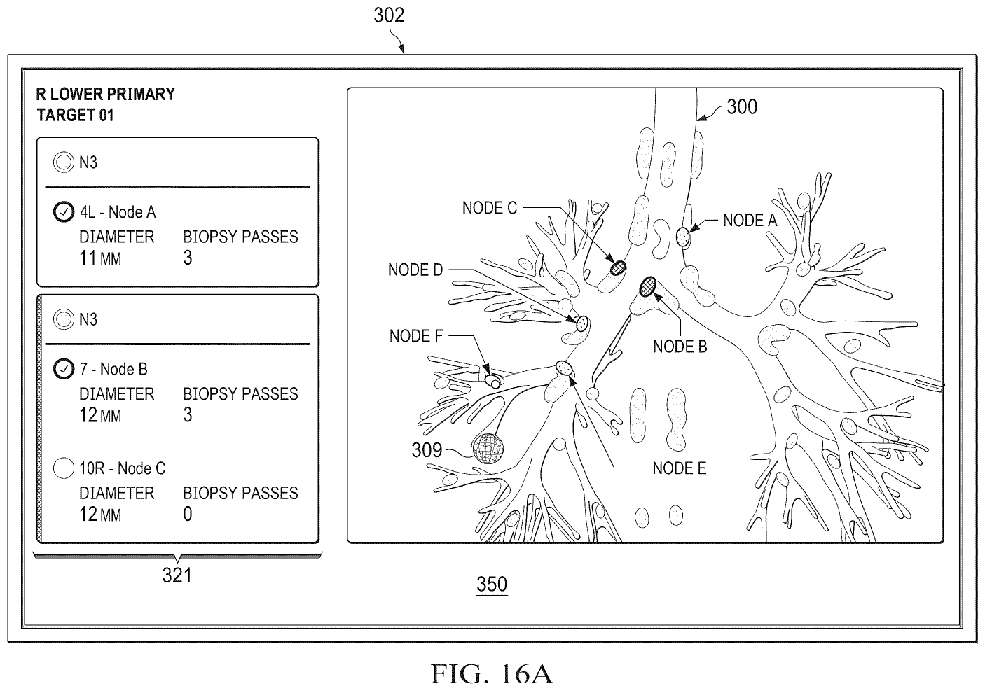

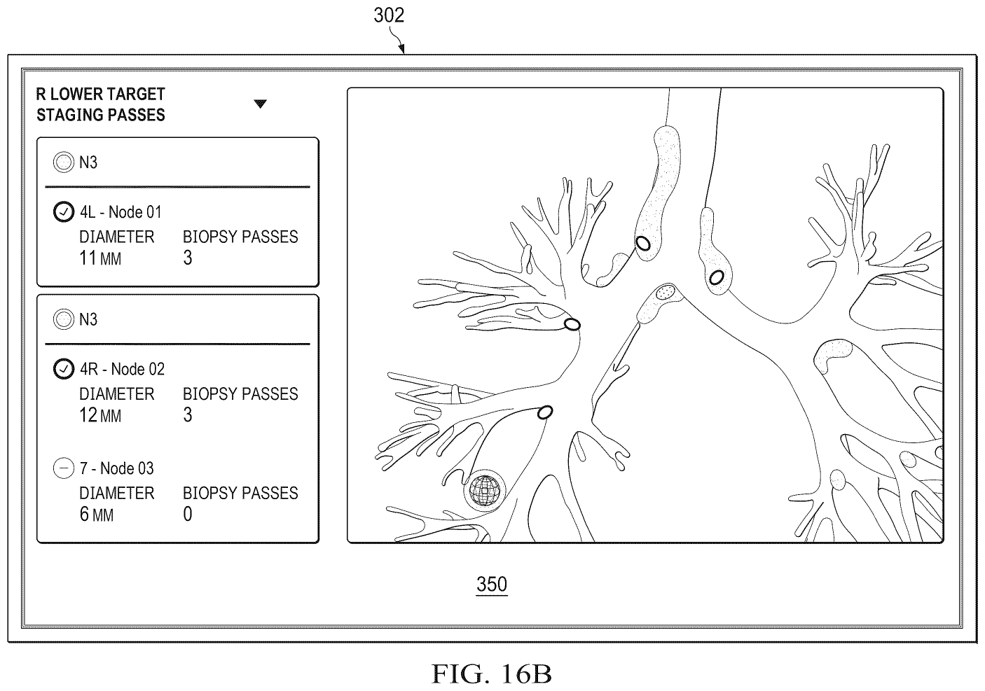

At a process 214, optionally, information from a clinical interventions at the sequenced nodes may be recorded and documented. This staging history information may include automated documentation of nodal information, procedure steps, and biopsy passes. For example, a clinical intervention, such as a biopsy procedure, may be performed at one or more nodal sites in the order of the sequence, and information about the biopsy may be recorded and associated with each node site. Biopsy information may include, for example, the number of biopsy passes, the pathology results for the samples, the depth of the biopsy needle, the volume of removed material or other information about the biopsy procedure. For example, and with reference to FIG. 16A, biopsy information may be recorded and stored for each biopsied node. The nodal information displayed in panel 321 may include, for example, the number of biopsy passes. For example, three biopsy passes were recorded for Node A, three biopsy passes were recorded for Node B and zero biopsy passes were recorded for Node C. FIG. 16B provides another example of a user interfaces 350, 352 that may be used.

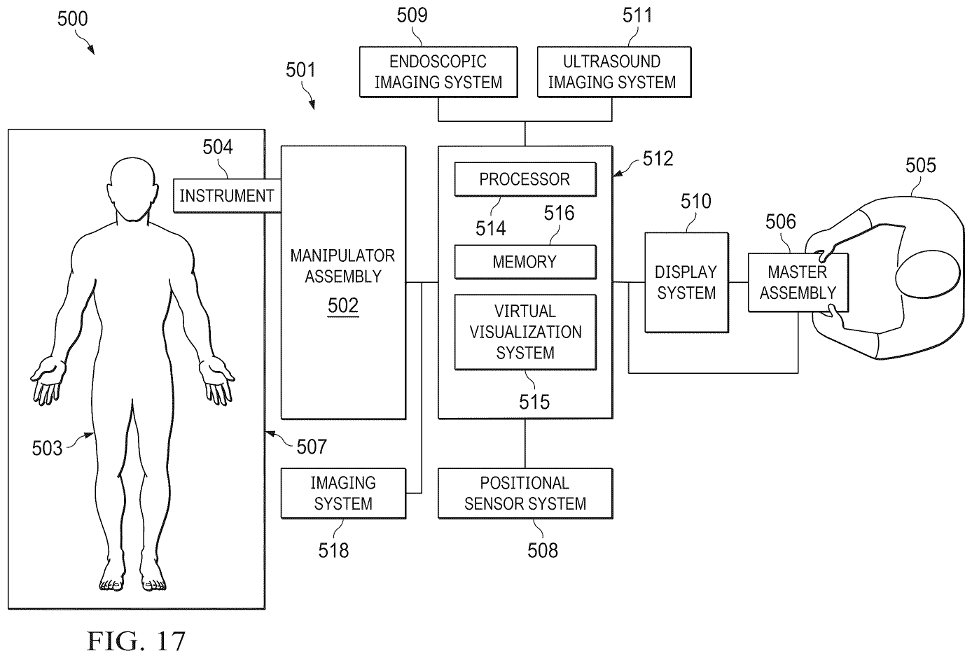

FIG. 17 is a schematic representation of a robotic or teleoperated medical system 500 (“medical system 500”) configured in accordance with various embodiments of the present technology. The medical system 500 can be used with any of the procedures or methods described above with respect to FIGS. 1A-16. For example, the medical system 500 can be used to plan and/or perform an imaging and/or biopsy procedure on one or more nodal sites, as previously discussed. As shown, the medical system 500 includes a manipulator assembly 502, a medical instrument system 504, a master assembly 506, and a control system 512. The manipulator assembly 502 supports the medical instrument system 504 and drives the medical instrument system 504 at the direction of the master assembly 506 and/or the control system 512 to perform various medical procedures on a patient 503 positioned on a table 507 in a surgical environment 501. In this regard, the master assembly 506 generally includes one or more control devices that can be operated by an operator 505 (e.g., a physician) to control the manipulator assembly 502. Additionally, or alternatively, the control system 512 includes a computer processor 514 and at least one memory 516 for effecting control between the medical instrument system 504, the master assembly 506, and/or other components of the medical system 500. The control system 512 can also include programmed instructions (e.g., a non-transitory computer-readable medium storing the instructions) to implement any one or more of the methods described herein, including instructions for providing information to a display system 510 and/or processing data for registration of the medical instrument system 504 with an anatomical model of the patient 503 (as described in greater detail below). The manipulator assembly 502 can be a teleoperated, a non-teleoperated, or a hybrid teleoperated and non-teleoperated assembly. Thus, all or a portion of the master assembly 506 and/or all or a portion of the control system 512 can be positioned inside or outside of the surgical environment 501.

To aid the operator 505 in controlling the manipulator assembly 502 and/or the medical instrument system 504 during an image-guided medical procedure, the medical system 500 may further include a positional sensor system 508, an endoscopic imaging system 509, an imaging system 518, and/or a virtual visualization system 515. In some embodiments, the positional sensor system 508 includes a location sensor system (e.g., an electromagnetic (EM) sensor system) and/or a shape sensor system for capturing positional sensor data (e.g., position, orientation, speed, velocity, pose, shape, etc.) of the medical instrument system 504. In these and other embodiments, the endoscopic imaging system 509 includes one or more image capture devices (not shown) that record endoscopic image data that includes concurrent or real-time images (e.g., video, still images, etc.) of patient anatomy. Images captured by the endoscopic imaging system 509 may be, for example, 2D or 3D images of patient anatomy captured by an image capture device positioned within the patient 503, and are referred to hereinafter as “real navigational images.”

In some embodiments, the medical instrument system 504 may include components of the positional sensor system 508 and/or components of the endoscopic imaging system 509. For example, components of the positional sensor system 508 and/or components of the endoscopic imaging system 509 can be integrally or removably coupled to the medical instrument system 504. Additionally, or alternatively, the endoscopic imaging system 509 can include a separate endoscope (not shown) attached to a separate manipulator assembly (not shown) that can be used in conjunction with the medical instrument system 504 to image patient anatomy. The positional sensor system 508 and/or the endoscopic imaging system 509 may be implemented as hardware, firmware, software, or a combination thereof that interact with or are otherwise executed by one or more computer processors, such as the computer processor(s) 514 of the control system 512.

In some examples, the medical instrument system 504 may include intra-operative imaging capabilities such as ultrasound imaging, for imaging tissue not visible with the endoscopic imaging system. Thus the medical instrument system 504 may include an ultrasound imaging system 511 including an ultrasound transducer for imaging tissue, such as lymph node, beyond the wall of the anatomic passage in which the instrument system is extended.

The imaging system 518 of the medical system 500 may be arranged in the surgical environment 501 near the patient 503 to obtain real-time and/or near real-time images of the patient 503 before, during, and/or after a medical procedure. In some embodiments, the imaging system 518 includes a mobile C-arm cone-beam CT imaging system for generating 3D images. For example, the imaging system 518 can include a DynaCT imaging system from Siemens Corporation, or another suitable imaging system. In these and other embodiments, the imaging system 518 can include other imaging technologies, including MRI, fluoroscopy, thermography, ultrasound, OCT, thermal imaging, impedance imaging, laser imaging, nanotube X-ray imaging, and/or the like.

The virtual visualization system 515 of the control system 512 provides navigation and/or anatomy-interaction assistance to the operator 505 when controlling the medical instrument system 504 during an image-guided medical procedure. As described in greater detail below, virtual navigation using the virtual visualization system 515 can be based, at least in part, upon reference to an acquired pre-operative or intra-operative dataset (e.g., based, at least in part, upon reference to data generated by the positional sensor system 508, the endoscopic imaging system 509, and/or the imaging system 518) of anatomic passageways of the patient 503. In some implementations, for example, the virtual visualization system 515 processes preoperative and/or intraoperative image data of an anatomic region of the patient 503 captured by the imaging system 518 to generate an anatomic model (not shown) of the anatomic region. The virtual visualization system 515 then registers the anatomic model to positional sensor data generated by the positional sensor system 508 and/or to endoscopic image data generated by the endoscopic imaging system 509 to (i) map the tracked position, orientation, pose, shape, and/or movement of the medical instrument system 504 within the anatomic region to a correct position within the anatomic model, and/or (ii) determine a virtual navigational image of virtual patient anatomy of the anatomic region from a viewpoint of the medical instrument system 504 at a location within the anatomic model corresponding to a location of the medical instrument system 504 within the patient 503.

The display system 510 (e.g. display system 302) may display various images or representations of patient anatomy and/or of the medical instrument system 504 that are generated by the positional sensor system 508, by the endoscopic imaging system 509, by the imaging system 518, and/or by the virtual visualization system 515. In some embodiments, the display system 510 and/or the master assembly 506 may be oriented so the operator 505 can control the manipulator assembly 502, the medical instrument system 504, the master assembly 506, and/or the control system 512 with the perception of telepresence.

As discussed above, the manipulator assembly 502 drives the medical instrument system 504 at the direction of the master assembly 506 and/or the control system 512. In this regard, the manipulator assembly 502 can include select degrees of freedom of motion that may be motorized and/or teleoperated and select degrees of freedom of motion that may be non-motorized and/or non-teleoperated. For example, the manipulator assembly 502 can include a plurality of actuators or motors (not shown) that drive inputs on the medical instrument system 504 in response to commands received from the control system 512. The actuators can include drive systems (not shown) that, when coupled to the medical instrument system 504, can advance the medical instrument system 504 into a naturally or surgically created anatomic orifice. Other drive systems may move a distal portion (not shown) of the medical instrument system 504 in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, or alternatively, the actuators can be used to actuate an articulable end effector of the medical instrument system 504 (e.g., for grasping tissue in the jaws of a biopsy device and/or the like).

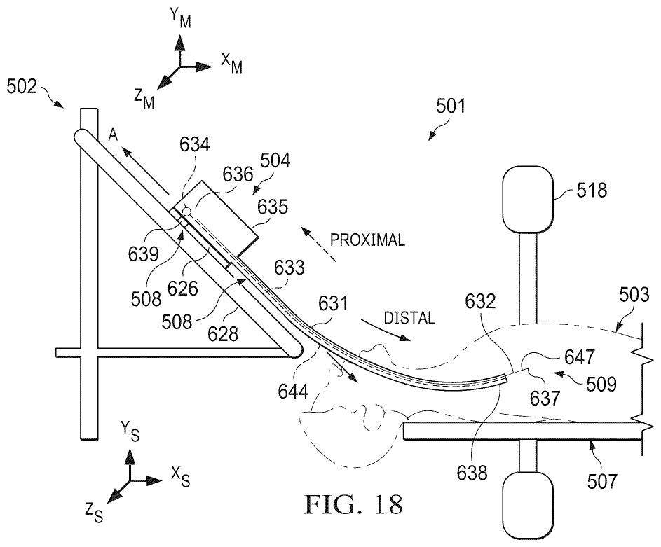

FIG. 18 is a schematic representation of the manipulator assembly 502, the medical instrument system 504, and the imaging system 518 of FIG. 17 within the surgical environment 501 and configured in accordance with various embodiments of the present technology. As shown in FIG. 18, the surgical environment 501 has a surgical frame of reference (XS, YS, ZS) in which the patient 503 is positioned on the table 507, and the medical instrument system 504 has a medical instrument frame of reference (XM, YM, ZM) within the surgical environment 501. During the medical procedure, the patient 503 may be stationary within the surgical environment 501 in the sense that gross patient movement can be limited by sedation, restraint, and/or other means. In these and other embodiments, cyclic anatomic motion of the patient 503, including respiration and cardiac motion, may continue unless the patient 503 is asked to hold his or her breath to temporarily suspend respiratory motion.

The manipulator assembly 502 includes an instrument carriage 626 mounted to an insertion stage 628. In the illustrated embodiment, the insertion stage 628 is linear, while in other embodiments, the insertion stage 628 is curved or has a combination of curved and linear sections. In some embodiments, the insertion stage 628 is fixed within the surgical environment 501. Alternatively, the insertion stage 628 can be movable within the surgical environment 501 but have a known location (e.g., via a tracking sensor (not shown) or other tracking device) within the surgical environment 501. In these alternatives, the medical instrument frame of reference (XM, YM, ZM) is fixed or otherwise known relative to the surgical frame of reference (XS, YS, ZS).

The medical instrument system 504 of FIG. 18 includes an elongate device 631, a medical instrument 632, an instrument body 635, at least a portion of the positional sensor system 508, and at least a portion of the endoscopic imaging system 509. In some embodiments, the elongate device 631 is a flexible catheter or other biomedical device that defines a channel or lumen 644. The channel 644 can be sized and shaped to receive the medical instrument 632 (e.g., via a proximal end 636 of the elongate device 631 and/or an instrument port (not shown)) and facilitate delivery of the medical instrument 632 to a distal portion 638 of the elongate device 631. The elongate device 631 is coupled to the instrument body 635, which in turn is coupled and fixed relative to the instrument carriage 626 of the manipulator assembly 502.

In operation, the manipulator assembly 502 can control insertion motion (e.g., proximal and/or distal motion along an axis A) of the elongate device 631 into the patient 503 via a natural or surgically created anatomic orifice of the patient 503 to facilitate navigation of the elongate device 631 through anatomic passageways of an anatomic region of the patient 503 and/or to facilitate delivery of a distal portion 638 of the elongate device 631 to or near a target location within the patient 503. For example, the instrument carriage 626 and/or the insertion stage 628 may include actuators (not shown), such as servomotors, that facilitate control over motion of the instrument carriage 626 along the insertion stage 628. Additionally, or alternatively, the manipulator assembly 502 in some embodiments can control motion of the distal portion 638 of the elongate device 631 in multiple directions, including yaw, pitch, and roll rotational directions (e.g., to navigate patient anatomy). To this end, the elongate device 631 may house or include cables, linkages, and/or other steering controls (not shown) that the manipulator assembly 502 can use to controllably bend the distal portion 638 of the elongate device 631. For example, the elongate device 631 can house at least four cables that can be used by the manipulator assembly 502 to provide (i) independent “up-down” steering to control a pitch of the distal portion 638 of the elongate device 631 and (ii) independent “left-right” steering of the elongate device 631 to control a yaw of the distal portion 638 of the elongate device 631.

The medical instrument 632 of the medical instrument system 504 can be used for medical procedures, such as for survey of anatomic passageways, surgery, biopsy, ablation, illumination, irrigation, and/or suction. Thus, the medical instrument 632 can include image capture probes, biopsy instruments or devices (e.g., biopsy needles, endobronchial ultrasound (EBUS) probes), laser ablation fibers, and/or other surgical, diagnostic, and/or therapeutic tools. For example, the medical instrument 632 can include an endoscope or other biomedical device having one or more image capture devices 647 positioned at a distal portion 637 of and/or at other locations along the medical instrument 632. In these embodiments, an image capture device 647 can capture one or more real navigational images or video (e.g., a sequence of one or more real navigational image frames) of anatomic passageways and/or other real patient anatomy while the medical instrument 632 is within an anatomic region of the patient 503.

As discussed above, the medical instrument 632 can be deployed into and/or be delivered to a target location within the patient 503 via the channel 644 defined by the elongate device 631. In embodiments in which the medical instrument 632 includes an endoscope or other biomedical device having an image capture device 647 at its distal portion 637, the image capture device 647 can be advanced to the distal portion 638 of the elongate device 631 before, during, and/or after the manipulator assembly 502 navigates the distal portion 638 of the elongate device 631 to a target location within the patient 503. In these embodiments, the medical instrument 632 can be used as a survey instrument to capture real navigational images of anatomic passageways and/or other real patient anatomy, to capture introperative ultrasound images of tissue beyond the anatomic passageway, and/or to aid an operator (not shown) to navigate the distal portion 638 of the elongate device 631 through anatomic passageways to the target location to perform an imaging procedure or a clinical intervention.

As another example, after the manipulator assembly 502 positions the distal portion 638 of the elongate device 631 proximate a target location within the patient 503, the medical instrument 632 can be advanced beyond the distal portion 638 of the elongate device 631 to perform a medical procedure at the target location. Continuing with this example, after all or a portion of the medical procedure at the target location is complete, the medical instrument 632 can be retracted back into the elongate device 631 and, additionally or alternatively, be removed from the proximal end 636 of the elongate device 631 or from another instrument port (not shown) along the elongate device 631.

As shown in FIG. 18, the positional sensor system 508 of the medical instrument system 504 includes a shape sensor 633 and a position measuring device 639. In these and other embodiments, the positional sensor system 508 can include other position sensors (e.g., accelerometers, rotary encoders, etc.) in addition to or in lieu of the shape sensor 633 and/or the position measuring device 639.

The shape sensor 633 of the positional sensor system 508 includes an optical fiber extending within and aligned with the elongate device 631. In one embodiment, the optical fiber of the shape sensor 633 has a diameter of approximately 200 μm. In other embodiments, the diameter of the optical fiber may be larger or smaller. The optical fiber of the shape sensor 633 forms a fiber optic bend sensor that is used to determine a shape, orientation, and/or pose of the elongate device 631. In some embodiments, optical fibers having Fiber Bragg Gratings (FBGs) can be used to provide strain measurements in structures in one or more dimensions. Various systems and methods for monitoring the shape and relative position of an optical fiber in three dimensions are described in further detail in U.S. Patent Application Publication No. 2006/0013523 (filed Jul. 13, 2005) (disclosing fiber optic position and shape sensing device and method relating thereto); U.S. Pat. No. 7,781,724 (filed on Sep. 26, 2006) (disclosing fiber-optic position and shape sensing device and method relating thereto); U.S. Pat. No. 7,772,541 (filed on Mar. 12, 2008) (disclosing fiber-optic position and/or shape sensing based on Rayleigh scatter); and U.S. Pat. No. 6,389,187 (filed on Jun. 17, 1998) (disclosing optical fiber bend sensors), which are all incorporated by reference herein in their entireties. In these and other embodiments, sensors of the present technology may employ other suitable strain sensing techniques, such as Rayleigh scattering, Raman scattering, Brillouin scattering, and Fluorescence scattering. In these and still other embodiments, the shape of the elongate device 631 may be determined using other techniques. For example, a history of the pose of the distal portion 638 of the elongate device 631 can be used to reconstruct the shape of elongate device 631 over an interval of time.

In some embodiments, the shape sensor 633 is fixed at a proximal point 634 on the instrument body 635 of the medical instrument system 504. In operation, for example, the shape sensor 633 measures a shape in the medical instrument reference frame (XM, YM, ZM) from the proximal point 634 to another point along the optical fiber, such as the distal portion 638 of the elongate device 631. The proximal point 634 of the shape sensor 633 may be movable along with instrument body 635 but the location of proximal point 634 may be known (e.g., via a tracking sensor (not shown) or other tracking device).

The position measuring device 639 of the positional sensor system 508 provides information about the position of the instrument body 635 as it moves along the insertion axis A on the insertion stage 628 of the manipulator assembly 502. In some embodiments, the position measuring device 639 includes resolvers, encoders, potentiometers, and/or other sensors that determine the rotation and/or orientation of actuators (not shown) controlling the motion of the instrument carriage 626 of the manipulator assembly 502 and, consequently, the motion of the instrument body 635 of the medical instrument system 504.

The systems and methods described herein can be provided in the form of tangible and non-transitory machine-readable medium or media (such as a hard disk drive, hardware memory, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

Although many of the embodiments are described above in the context of navigating and performing medical procedures within lungs of a patient, other applications and other embodiments in addition to those described herein are within the scope of the present technology. For example, unless otherwise specified or made clear from context, the devices, systems, methods, and computer program products of the present technology can be used for various image-guided medical procedures, such as medical procedures performed on, in, or adjacent hollow patient anatomy, and, more specifically, in procedures for surveying, biopsying, ablating, or otherwise treating tissue within and/or proximal the hollow patient anatomy. Thus, for example, the systems, devices, methods, and computer program products of the present disclosure can be used in one or more medical procedures associated with other patient anatomy, such as the bladder, urinary tract, GI system, and/or heart of a patient.

As used herein, the term “operator” shall be understood to include any type of personnel who may be performing or assisting a medical procedure and, thus, is inclusive of a physician, a surgeon, a doctor, a nurse, a medical technician, other personnel or user of the technology disclosed herein, and any combination thereof. Additionally, or alternatively, the term “patient” should be considered to include human and/or non-human (e.g., animal) patients upon which a medical procedure is being performed.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. As another example, various components of the technology can be further divided into subcomponents, and/or various components and/or functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology.

It should also be noted that other embodiments in addition to those disclosed herein are within the scope of the present technology. For example, embodiments of the present technology can have different configurations, components, and/or procedures in addition to those shown or described herein. Moreover, a person of ordinary skill in the art will understand that these and other embodiments can be without several of the configurations, components, and/or procedures shown or described herein without deviating from the present technology. Accordingly, the disclosure and associated technology can encompass other embodiments not expressly shown or described herein.

1. A system for providing navigational guidance for a medical procedure, the system comprising:
a processor; and
a memory operably coupled to the processor and storing instructions that, when executed by the processor, cause the system to perform operations comprising:
receiving an anatomic model of an anatomic region;
performing an analysis of the anatomic model to facilitate selection of a plurality of nodal sites for analysis;
generating a procedure sequence for the plurality of nodal sites;
providing navigational guidance to direct a medical instrument to a nodal site of the plurality of nodal sites in the procedure sequence; and
gathering local image data from the medical instrument at the nodal site.

2. The system of claim 1, wherein performing the analysis includes identifying a plurality of anatomic stations for the anatomic model.

3. The system of claim 1, wherein performing the analysis includes identifying a plurality of lymph nodes in the anatomic model.

4. The system of claim 3, wherein the performed operations further comprise displaying on a graphical user interface an image of the anatomic model and an image of a segmented lymph node of the plurality of lymph nodes.

5. The system of claim 4, wherein the image of the segmented lymph node is overlayed on an image of an anatomic station of the anatomic model.

6. The system of claim 5, wherein the performed operations further comprise highlighting the image of the segmented lymph node if a staging threshold is met.

7. The system of claim 4, wherein the performed operations further comprise overlaying PET imaging information on the image of the anatomic model.

8. The system of claim 3, wherein the navigational guidance includes a displayed list view of the plurality of lymph nodes grouped by a plurality of anatomic stations for the anatomic model.

9. The system of claim 8, wherein the list view includes a size or a shape for at least one of the plurality of lymph nodes.

10. (canceled)

11. The system of claim 3 wherein the performed operations further comprise displaying critical anatomy near at least one of the plurality of nodal sites.

12. The system of claim 1, wherein the navigational guidance includes an image of the anatomic model and a representation of a navigable path through the procedure sequence for the plurality of nodal sites.

13. The system of claim 12, wherein the representation of the navigable path includes a first portion indicating a portion of the navigable path that has been reached by the medical instrument and a second portion indicating a portion of the navigable path that has not been reached by the medical instrument and wherein the first portion is visually distinguishable from the second portion.

14. The system of claim 12, wherein the navigational guidance further includes a displayed representation of critical anatomy along the navigable path or displayed nodal markers adjacent to one or more of the plurality of nodal sites.

15. (canceled)

16. The system of claim 1, wherein the navigational guidance includes an indicator configured to indicate that at least one nodal site of the plurality of nodal sites has or has not been sampled by the medical instrument.

17. (canceled)

18. The system of claim 1, wherein the navigational guidance includes an indicator configured to indicate a nodal site of the plurality of nodal sites to be sampled by the medical instrument.

19. The system of claim 18, wherein the indicator is displayed with a three-dimensional image of the anatomic model.

20. The system of claim 18, wherein the indicator is displayed with a virtual or a real-time endoscopic image view.

21. (canceled)

22. The system of claim 1, wherein providing navigational guidance includes displaying a user interface orientation element including a direction indicator for an imaging element of the medical instrument and a representation of a critical anatomy.

23. The system of claim 1, wherein providing navigational guidance includes displaying a representation of a field of view area of an imaging element of the medical instrument.

24. The system of claim 1, wherein the performed operations further comprise displaying the local image data with an image annotation of the local image data.

25-62. (canceled)
